(19) United States
(12) Patent Application Publication
(10) Pub. No.: US 2011/0287405 A1
(43) Pub. Date: Nov. 24, 2011

(54) BIOMASS PRODUCTION

(75) Inventors: Jaime A. Gonzalez, Oakville (CA); Max Kolesnik, Schomberg (CA); Steven C. Martin, Toronto (CA); Tony Dipietro, Brampton (CA); Emidio Dipietro, Bampton (CA)

(73) Assignee: POND BIOFUELS INC., Scarborough (CA)

(21) Appl. No.: 13/022,396

(22) Filed: Feb. 7, 2011

Related U.S. Application Data
(63) Continuation-in-part of application No. 12/784,172, filed on May 20, 2010.

(51) Int. Cl. C12Q 3/00; C12N 1/00; C12N 5/04

(52) U.S. Cl. 435/3; 435/410; 435/243

(57) ABSTRACT

There is provided a process of growing a phototrophic biomass in a reaction zone. The reaction zone comprises a production purpose reaction mixture that is operative for effecting photosynthesis upon exposure to photosynthetically active light radiation. The production purpose reaction mixture comprises production purpose phototrophic biomass that is operative for growth within the reaction zone, such that a reaction zone concentration of production purpose phototrophic biomass is provided in the reaction zone. The growth of the production purpose phototrophic biomass comprises that which is effected by the photosynthesis.

### CROSS-REFERENCE TO RELATED APPLICATIONS

This application is a continuation-in-part of U.S. application Ser. No. 12/784,172, filed on May 20, 2010, which is incorporated herein by reference in its entirety.

### TECHNICAL FIELD

The present disclosure relates to a process for growing biomass.

### BACKGROUND

The cultivation of phototrophic organisms has been widely practiced for purposes of producing a fuel source. Exhaust gases from industrial processes have also been used to promote the growth of phototrophic organisms by supplying carbon dioxide for consumption by phototrophic organisms during photosynthesis. By providing exhaust gases for such purpose, environmental impact is reduced and, in parallel a potentially useful fuel source is produced. Challenges remain, however, to render this approach more economically attractive for incorporation within existing facilities.

### SUMMARY

In one aspect, there is provided a process of growing a phototrophic biomass in a reaction zone. The reaction zone comprises a production purpose reaction mixture that is operative for effecting photosynthesis upon exposure to photosynthetically active light radiation. The production purpose reaction mixture comprises production purpose phototrophic biomass that is operative for growth within the reaction zone, such that a reaction zone concentration of production purpose phototrophic biomass is provided in the reaction zone. The growth of the production purpose phototrophic biomass comprises that which is effected by the photosynthesis. While effecting growth of the production purpose phototrophic biomass in the reaction zone, and while supplying aqueous feed material to the reaction zone and discharging reaction zone product from the reaction zone, wherein the reaction zone product comprises a portion of the production purpose phototrophic biomass: when a sensed value of a process parameter is different than a target value of the process parameter, modulating the molar rate of discharge of the reaction zone product from the reaction zone, wherein the target value of the process parameter is based upon a desired growth rate of the production purpose phototrophic biomass.

In another aspect, there is provided another process of growing a phototrophic biomass in a reaction zone. The reaction zone comprises a production purpose reaction mixture that is operative for effecting photosynthesis upon exposure to photosynthetically active light radiation. The production purpose reaction mixture comprises production purpose phototrophic biomass that is operative for growth within the reaction zone. The growth of the production purpose phototrophic biomass comprises that which is effected by the photosynthesis. While effecting growth of the production purpose phototrophic biomass within the reaction zone at a rate that exceeds 90% of the maximum molar growth rate of the production purpose phototrophic biomass within the reaction zone, a reaction zone product including production purpose phototrophic biomass is discharged from the reaction zone to provide a molar rate of discharge of the production purpose phototrophic biomass that is at least 90% of the maximum molar growth rate of the production purpose phototrophic biomass within the reaction zone.

### BRIEF DESCRIPTION OF THE DRAWINGS

The process of the preferred embodiments of the invention will now be described with the following accompanying drawings:

FIG. 1 is a process flow diagram of an embodiment of the process;

FIG. 2 is a process flow diagram of another embodiment of the process; and

FIG. 3 is a schematic illustration of a portion of a fluid passage of an embodiment of the process.

### DETAILED DESCRIPTION

Reference throughout the specification to “some embodiments” means that a particular feature, structure, or characteristic described in connection with some embodiments are not necessarily referring to the same embodiments. Furthermore, the particular features, structure, or characteristics may be combined in any suitable manner with one another.

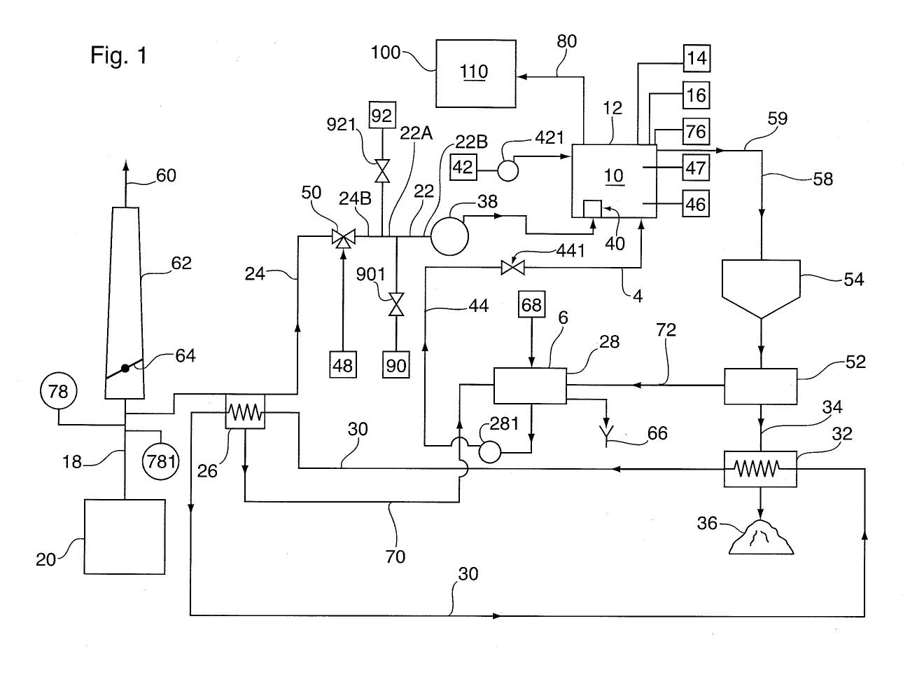

Referring to FIG. 1, there is provided a process of growing a phototrophic biomass in a reaction zone 10. The reaction zone 10 includes a reaction mixture that is operative for effecting photosynthesis upon exposure to photosynthetically active light radiation. The reaction mixture includes phototrophic biomass material, carbon dioxide, and water. In some embodiments, the reaction zone includes phototrophic biomass and carbon dioxide disposed in an aqueous medium. Within the reaction zone, the phototrophic biomass is disposed in mass transfer communication with both of carbon dioxide and water. In some embodiments, for example, the reaction mixture includes phototrophic biomass disposed in an aqueous medium, and carbon dioxide-enriched phototrophic biomass is provided upon the receiving of carbon dioxide by the phototrophic biomass.

“Phototrophic organism” is an organism capable of phototrophic growth in the aqueous medium upon receiving light energy, such as plant cells and micro-organisms. The phototrophic organism is unicellular or multicellular. In some embodiments, for example, the phototrophic organism is an organism which has been modified artificially or by gene manipulation. In some embodiments, for example, the phototrophic organism is an alga. In some embodiments, for example, the algae are microalgae.

“Phototrophic biomass” is at least one phototrophic organism. In some embodiments, for example, the phototrophic biomass includes more than one species of phototrophic organisms.

“Reaction zone 10” defines a space within which the growing of the phototrophic biomass is effected. In some embodiments, for example, the reaction zone 10 is provided in a photobioreactor 12. In some embodiments, for example, pressure within the reaction zone is atmospheric pressure.

“Photobioreactor 12” is any structure, arrangement, land formation or area that provides a suitable environment for the growth of phototrophic biomass. Examples of specific structures which can be used is a photobioreactor 12 by providing space for growth of phototrophic biomass using light energy include, without limitation, tanks, ponds, troughs, ditches, pools, pipes, tubes, canals, and channels. Such photobioreactors may be either open, closed, partially closed, covered, or partially covered. In some embodiments, for example, the photobioreactor 12 is a pond, and the pond is open, in which case the pond is susceptible to uncontrolled receiving of materials and light energy from the immediate environments. In other embodiments, for example, the photobioreactor 12 is a covered pond or a partially covered pond, in which case the receiving of materials from the immediate environment is at least partially interfered with. The photobioreactor 12 includes the reaction zone 10 which includes the reaction mixture. In some embodiments, the photobioreactor 12 is configured to receive a supply of phototrophic reagents (and, in some of these embodiments, optionally, supplemental nutrients), and is also configured to effect discharge of phototrophic biomass which is grown within the reaction zone 10. In this respect, in some embodiments, the photobioreactor 12 includes one or more inlets for receiving the supply of phototrophic reagents and supplemental nutrients, and also includes one or more outlets for effecting the recovery or harvesting of biomass which is grown within the reaction zone 10. In some embodiments, for example, one or more of the inlets are configured to be temporarily sealed for periodic or intermittent time intervals. In some embodiments, for example, one or more of the outlets are configured to be temporarily sealed or substantially sealed for periodic or intermittent time intervals. The photobioreactor 12 is configured to contain the reaction mixture which is operative for effecting photosynthesis upon exposure to photosynthetically active light radiation. The photobioreactor 12 is also configured so as to establish photosynthetically active light radiation (for example, a light of a wavelength between about 400-700 nm, which can be emitted by the sun or another light source) within the photobioreactor 12 for exposing the phototrophic biomass. The exposing of the reaction mixture to the photosynthetically active light radiation effects photosynthesis and growth of the phototrophic biomass. In some embodiments, for example, the established light radiation is provided by an artificial light source 14 disposed within the photobioreactor 12. For example, suitable artificial lights sources include submersible fiber optics or light guides, light-emitting diodes (“LEDs”), LED strips and fluorescent lights. Any LED strips known in the art can be adapted for use in the photobioreactor 12. In the case of the submersible LEDs, in some embodiments, for example, energy sources include alternative energy sources, such as wind, photovoltaic cells, fuel cells, etc. to supply electricity to the LEDs. Fluorescent lights, external or internal to the photobioreactor 12, can be used as a back-up system. In some embodiments, for example, the established light is derived from a natural light source 16 which has been transmitted from externally of the photobioreactor 12 and through a transmission component. In some embodiments, for example, the transmission component is a portion of a containment structure of the photobioreactor 12 which is at least partially transparent to the photosynthetically active light radiation, and which is configured to provide for transmission of such light to the reaction zone 10 for receiving by the phototrophic biomass. In some embodiments, for example, natural light is received by a solar collector, filtered with selective wavelength filters, and then transmitted to the reaction zone 10 with fiber optic material or with a light guide. In some embodiments, for example, both natural and artificial lights sources are provided for effecting establishment of the photosynthetically active light radiation within the photobioreactor 12.

“Aqueous medium” is an environment that includes water. In some embodiments, for example, the aqueous medium also includes sufficient nutrients to facilitate viability and growth of the phototrophic biomass. In some embodiments, for example, supplemental nutrients may be included such as one of, or both of, NO_{X} and SO_{X}. Suitable aqueous media are discussed in detail in: Rogers, L. J. and Gallon J. R. “Biochemistry of the Algae and Cyanobacteria,” Clarendon Press Oxford, 1988; Burlew, John S. “Algal Culture: From Laboratory to Pilot Plant.” Carnegie Institution of Washington Publication 600. Washington, D.C., 1961 (hereinafter “Burlew 1961”); and Round, F. E. The Biology of the Algae. St Martin's Press, New York, 1965; each of which is incorporated herein by reference). A suitable supplemental nutrient composition, known as “Bold's Basal Medium”, is described in Bold, H. C. 1949, The morphology of Chlamydomonas chlamydogama sp. nov. Bull. Torrey Bot. Club. 76: 101-8 (see also Bischoff, H. W. and Bold, H. C. 1963, Phycological Studies IV. Some soil algae from Enchanted Rock and related algal species, Univ. Texas Publ. 6318: 1-95, and Stein J. (ED.) Handbook of Phycological Methods, Culture methods and growth measurements, Cambridge University Press, pp. 7-24).

“Modulating”, with respect to a process parameter, such as an input or output, means any one of initiating, terminating, increasing, decreasing, or otherwise changing the process parameter, such as that of an input or an output.

In some embodiments, the process includes supplying the reaction zone 10 with carbon dioxide. In some of these embodiments, for example, the carbon dioxide supplied to the reaction zone 10 is derived from a gaseous exhaust material 18. In this respect, in some embodiments, the carbon dioxide is supplied by a gaseous exhaust material producing process 20, and the supplying is, therefore, effected by producing the gaseous exhaust material 18 with a gaseous exhaust material producing process 20. The gaseous exhaust material 18 includes carbon dioxide. The gaseous exhaust material producing process 20 includes any process which effects production of the gaseous exhaust material 18. In some embodiments, for example, the gaseous exhaust material producing process 20 is a combustion process. In some embodiments, for example, the combustion process is effected in a combustion facility. In some of these embodiments, for example, the combustion process effects combustion of a fossil fuel, such as coal, oil, or natural gas. For example, the combustion facility is any one of a fossil fuel-fired power plant, an industrial incineration facility, an industrial furnace, an industrial heater, or an internal combustion engine. In some embodiments, for example, the combustion facility is a cement kiln.

Reaction zone feed material 22 is supplied to the reaction zone 10 such that carbon dioxide of the reaction zone feed material 22 is received within the reaction zone 10. During at least some periods of operation of the process, at least a fraction of the reaction zone feed material 22 is supplied by the gaseous exhaust material 18 which is discharged from the gaseous exhaust material producing process 20. Any of the gaseous exhaust material 18 that is supplied to the reaction zone feed material 22 is supplied as a gaseous exhaust material reaction zone supply 24. It is understood that not the entirety of the gaseous exhaust material 18 is necessarily supplied to the gaseous exhaust material reaction zone supply 24, or at least not for the entire time period during which the process is operational. The gaseous exhaust material reaction zone supply 24 includes carbon dioxide. In some embodiments, for example, the gaseous exhaust material 18 includes a carbon dioxide concentration of at least 2 volume % based on the total volume of the gaseous exhaust material 18. In this respect, in some embodiments, for example, the gaseous exhaust material reaction zone supply 24 includes a carbon dioxide concentration of at least 2 volume % based on the total volume of the gaseous exhaust material reaction zone supply 24. In some embodiments, for example, the gaseous exhaust material 18 includes a carbon dioxide concentration of at least 4 volume % based on the total volume of the gaseous exhaust material 18. In this respect, in some embodiments, for example, the gaseous exhaust material reaction zone supply 24 includes a carbon dioxide concentration of at least 4 volume % based on the total volume of the gaseous exhaust material reaction zone supply 24. In some embodiments, for example, the gaseous exhaust material reaction zone supply 24 also includes one of, or both of, NO_{X} and SO_{X}.

In some of these embodiments, for example, the gaseous exhaust material reaction zone supply 24 is at least a fraction of the gaseous exhaust material 18 being produced by the gaseous exhaust material producing process 20. In some cases, the entirety of the gaseous exhaust material 18 produced by the gaseous exhaust material producing process 20 is supplied to the gaseous exhaust material reaction zone supply 24.

In some embodiments, for example, the reaction zone feed material 22 is cooled prior to supply to the reaction zone 10 so that the temperature of the reaction zone feed material 22 aligns with a suitable temperature at which the phototrophic biomass can grow. In some embodiments, for example, the gaseous exhaust material reaction zone supply 24 being supplied to the reaction zone material 22 is disposed at a temperature of between 110 degrees Celsius and 150 degrees Celsius. In some embodiments, for example, the temperature of the gaseous exhaust material reaction zone supply 24 is about 132 degrees Celsius. In some embodiments, the temperature at which the gaseous exhaust material reaction zone supply 24 is disposed is much higher than this, and, in some embodiments, such as the gaseous exhaust material reaction zone supply 24 from a steel mill, the temperature is over 500 degrees Celsius. In some embodiments, for example, the gaseous exhaust material reaction zone supply 24 is cooled to between 20 degrees Celsius and 50 degrees Celsius (for example, about 30 degrees Celsius), either directly, or as a component of the reaction zone feed material 22 (as described above, the reaction zone feed material 22 is supplied with the gaseous exhaust material reaction zone supply 24). Supplying the reaction zone feed material 22 at higher temperatures could hinder growth, or even kill the phototrophic biomass in the reaction zone 10. In some of these embodiments, in effecting the cooling of the gaseous exhaust material reaction zone supply 24, at least a fraction of any water vapour in the gaseous exhaust material reaction zone supply 24 is condensed in a heat exchanger 26 (such as a condenser) and separated from the reaction zone feed material 22 as an aqueous material 70. In some embodiments, the resulting aqueous material 70 is diverted to a container 28 (described below) where it provides supplemental aqueous material supply 44 for supply to the reaction zone 10. In some embodiments, the condensing effects heat transfer from the reaction zone feed material 22 to a heat transfer medium 30, thereby raising the temperature of the heat transfer medium 30 to produce a heated heat transfer medium 30, and the heated heat transfer medium 30 is then supplied (for example, flowed) to a dryer 32 (discussed below), and heat transfer is effected from the heated heat transfer medium 30 to an intermediate concentrated reaction zone product 34 to effect drying of the intermediate concentrated reaction zone product 34 and thereby effect production of the final reaction zone product 36. In some embodiments, for example, after being discharged from the dryer 32, the heat transfer medium 30 is recirculated to the heat exchanger 26. Examples of a suitable heat transfer medium 30 include thermal oil and glycol solution.

With respect to the reaction zone feed material 22, the reaction zone feed material 22 is a fluid. In some embodiments, for example, the reaction zone feed material 22 is a gaseous material. In some embodiments, for example, the reaction zone feed material 22 includes gaseous material disposed in liquid material. In some embodiments, for example, the liquid material is an aqueous material. In some of these embodiments, for example, at least a fraction of the gaseous material is dissolved in the liquid material. In some of these embodiments, for example, at least a fraction of the gaseous material is disposed as a gas dispersion in the liquid material. In some of these embodiments, for example, and during at least some periods of operation of the process, the gaseous material of the reaction zone feed material 22 includes carbon dioxide supplied by the gaseous exhaust material reaction zone supply 24. In some of these embodiments, for example, the reaction zone feed material 22 is supplied to the reaction zone 10 as a flow.

In some embodiments, for example, the reaction zone feed material 22 is supplied to the reaction zone 10 as one or more reaction zone feed material flows. For example, each of the one or more reaction zone feed material flows is flowed through a respective reaction zone feed material fluid passage. In some of those embodiments where there are more than one reaction zone feed material flow, the material composition varies between the reaction zone feed material flows. In some embodiments, for example, a flow of reaction zone feed material 22 includes a flow of the gaseous exhaust material reaction zone feed material supply 24. In some embodiments, for example, a flow of reaction zone feed material 22 is a flow of the gaseous exhaust material reaction zone feed material supply 24.

In some embodiments, for example, the supply of the reaction zone feed material 22 to the reaction zone 10 effects agitation of at least a fraction of the phototrophic biomass disposed in the reaction zone 10. In this respect, in some embodiments, for example, the reaction zone feed material 22 is introduced to a lower portion of the reaction zone 10. In some embodiments, for example, the reaction zone feed material 22 is introduced from below the reaction zone 10 so as to effect mixing of the contents of the reaction zone 10. In some of these embodiments, for example, the effected mixing (or agitation) is such that any difference in phototrophic biomass concentration between two points in the reaction zone 10 is less than 20%. In some embodiments, for example, any difference in phototrophic biomass concentration between two points in the reaction zone 10 is less than 10%. In some of these embodiments, for example, the effected mixing is such that a homogeneous suspension is provided in the reaction zone 10. In those embodiments with a photobioreactor 12, for some of these embodiments, for example, the supply of the reaction zone feed material 22 is co-operatively configured with the photobioreactor 12 so as to effect the desired agitation of the at least a fraction of the phototrophic biomass disposed in the reaction zone 10.

With further respect to those embodiments where the supply of the reaction zone feed material 22 to the reaction zone 10 effects agitation of at least a fraction of the phototrophic biomass disposed in the reaction zone 10, in some of these embodiments, for example, the reaction zone feed material 22 flows through a gas injection mechanism, such as a sparger 40, before being introduced to the reaction zone 10. In some of these embodiments, for example, the sparger 40 provides reaction zone feed material 22 as a gas-liquid mixture, including fine gas bubbles entrained in a liquid phase, to the reaction zone 10 in order to maximize the interface contact area between the phototrophic biomass and the carbon dioxide (and, in some embodiments, for example, one of, or both of, SO_{X} and NO_{X}) of the reaction zone feed material 22. This assists the phototrophic biomass in efficiently absorbing the carbon dioxide (and, in some embodiments, other gaseous components) required for photosynthesis, thereby promoting the optimization of the growth rate of the phototrophic biomass. As well, in some embodiments, for example, the sparger 40 provides reaction zone feed material 22 in larger bubbles that agitate the phototrophic biomass in the reaction zone 10 to promote mixing of the components of the reaction zone 10. An example of a suitable sparger 40 is EDI FlexAir™ T-Series Tube Diffuser Model 91 X 1003 supplied by Environmental Dynamics Inc. of Columbia, Mo. In some embodiments, for example, this sparger 40 is disposed in a photobioreactor 12 having a reaction zone 10 volume of 6000 litres and with an algae concentration of between 0.8 grams per litre and 1.5 grams per litre, and the reaction zone feed material 22 is a gaseous fluid flow supplied at a flow rate of between 10 cubic feet per minute and 20 cubic feet per minute, and at a pressure of about 68 inches of water.

With respect to the sparger 40, in some embodiments, for example, the sparger 40 is designed to consider the fluid head of the reaction zone 10, so that the supplying of the reaction zone feed material 22 to the reaction zone 10 is effected in such a way as to promote the optimization of carbon dioxide absorption by the phototrophic biomass. In this respect, bubble sizes are regulated so that they are fine enough to promote optimal carbon dioxide absorption by the phototrophic biomass from the reaction zone feed material. Concomitantly, the bubble sizes are large enough so that at least a fraction of the bubbles rise through the entire height of the reaction zone 10, while mitigating against the reaction zone feed material 22 “bubbling through” the reaction zone 10 and being released without being absorbed by the phototrophic biomass. To promote the realization of an optimal bubble size, in some embodiments, the pressure of the reaction zone feed material 22 is controlled using a pressure regulator upstream of the sparger 40.

With respect to those embodiments where the reaction zone 10 is disposed in a photobioreactor 12, in some of these embodiments, for example, the sparger 40 is disposed externally of the photobioreactor 12. In other embodiments, for example, the sparger 40 is disposed within the photobioreactor 12. In some of these embodiments, for example, the sparger 40 extends from a lower portion of the photobioreactor 12 (and within the photobioreactor 12).

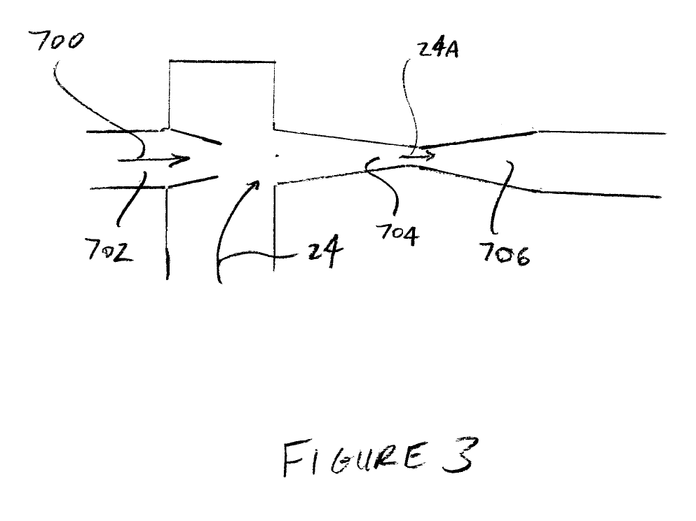

In some embodiments, for example, the reaction zone feed material 22 is supplied at a pressure which effects flow of the reaction zone feed material 22 through at least a seventy (70) inch vertical extent of the reaction zone. In some embodiments, for example, the vertical extent is at least 10 feet. In some embodiments, for example, the vertical extent is at least 20 feet. In some embodiments, for example, the vertical extent is at least 30 feet. In some of these embodiments, for example, the supplying of the reaction zone feed material 22 is effected while the gaseous exhaust material 18 is being produced by the gaseous exhaust material producing process 20 and while at least a fraction of the gaseous exhaust material 18 is being supplied to the reaction zone feed material 22 (as the gaseous exhaust material reaction zone supply 24). In some of these embodiments, for example, the pressure of the material of a flow of the gaseous exhaust material reaction zone supply 24 (whether by itself or as a portion of the flow of the reaction zone feed material 22) is increased before being supplied to the reaction zone 10. In some embodiments, for example, the pressure increase is at least partially effected by a prime mover 38. For those embodiments where the pressure increase is at least partially effected by the prime mover 38. An example of a suitable prime mover 38, for embodiments where the gaseous exhaust material reaction zone supply 24 is a portion of a flow of the reaction zone feed material 22, and the reaction zone feed material includes liquid material, is a pump. Examples of a suitable prime mover 38, for embodiments where the pressure increase is effected to a gaseous flow, include a blower, a compressor, and an air pump. In other embodiments, for example, the pressure increase is effected by a jet pump or eductor. With respect to such embodiments, where the pressure increase is effected by a jet pump or eductor, in some of these embodiments, for example, the gaseous exhaust material reaction zone supply 24 is supplied to the jet pump or eductor and pressure energy is transferred to the gaseous exhaust material reaction zone from another flowing fluid (the “motive fluid flow”) using the venturi effect to effect a pressure increase in the gaseous exhaust material reaction zone supply 24 component of the reaction zone feed material 22. In this respect, in some embodiments, for example, and referring to FIG. 3, a motive fluid flow 700 is provided, wherein material of the motive fluid flow 700 includes a motive fluid pressure P_{M1}, wherein P_{M1} is greater than the pressure (P_{E}) of the gaseous exhaust material reaction zone supply 24. Pressure of the motive fluid flow 700 is reduced from P_{M1} to P_{M2} by flowing the motive fluid flow 700 from an upstream fluid passage portion 702 to an intermediate downstream fluid passage portion 704. The first intermediate downstream fluid passage portion 704 is characterized by a smaller cross-sectional area relative to the upstream fluid passage portion 702. Further, P_{M2} is less than P_{E}. When the pressure of the motive fluid flow 700 has becomes reduced to P_{M2}, fluid communication between the motive fluid flow 700 and the gaseous exhaust material reaction zone supply 24 is effected such that the material of the gaseous exhaust material reaction zone supply 24 is induced to mix with the motive fluid flow 700 in the intermediate downstream fluid passage portion 704, in response to the pressure differential between the supply 24 and the motive fluid flow 700, to produce a gaseous exhaust material reaction zone supply-derived flow 24A. Pressure of the gaseous exhaust material reaction zone supply-derived flow 24A, which includes the gaseous exhaust material reaction zone supply is increased to P_{M3}, wherein P_{M3} is greater than P_{E}. The pressure increase is effected by flowing the gaseous exhaust material reaction zone supply-derived flow 24A from the intermediate downstream fluid passage portion 704 to a “kinetic energy to static pressure energy conversion” downstream fluid passage portion 706. The cross-sectional area of the “kinetic energy to static pressure energy conversion” downstream fluid passage portion 706 is greater than the cross-sectional area of the intermediate downstream fluid passage portion 704. The gaseous exhaust material reaction zone supply-derived flow 24A, including the gaseous exhaust material reaction zone supply 24, is disposed at a pressure that is greater than P_{E} and that is sufficient to effect flow of material of the flow 24A, as at least a portion of the flow of the reaction zone feed material 22, through at least a seventy (70) inch vertical extent of the reaction zone 10. In some embodiments, for example, a converging nozzle portion of a fluid passage defines the first intermediate downstream fluid passage portion 704 and a diverging nozzle portion of the fluid passage defines the “kinetic energy to static pressure energy conversion” downstream fluid passage portion 706. In some embodiments, for example, the combination of the first intermediate downstream fluid passage portion 704 and the “kinetic energy to static pressure energy conversion” downstream fluid passage portion 706 is defined by a venture nozzle. In some embodiments, for example, the combination of the first intermediate downstream fluid passage portion 704 and the “kinetic energy to static pressure energy conversion” downstream fluid passage portion 706 is disposed within an eductor or jet pump. In some of these embodiments, for example, the motive fluid flow includes liquid aqueous material and, in this respect, the flow 24A includes a combination of liquid and gaseous material. In this respect, in some embodiments, for example, the gaseous exhaust material reaction zone supply-derived flow 24A includes a dispersion of a gaseous material within a liquid material, wherein the dispersion of a gaseous material includes carbon dioxide of the gaseous exhaust material reaction zone supply 24. Alternatively, in some of these embodiments, for example, the motive fluid flow is another gaseous flow, such as an air flow, and the flow 24A is a gaseous flow. The material of the flow 24A is supplied to the reaction zone 10, as at least a portion of a flow of the reaction zone feed material 22, at a pressure greater than P_{E} and sufficient to effect flow of the material of the flow 24A through at least a seventy (70) inch vertical extent of the reaction zone 10. This pressure increase is designed to overcome the fluid head within the reaction zone 10.

In some embodiments, for example, the photobioreactor 12, or plurality of photobioreactors 12, are configured so as to optimize carbon dioxide absorption by the phototrophic biomass and reduce energy requirements. In this respect, the photobioreactor (s) is (are) configured to provide increased residence time of the carbon dioxide within the reaction zone 10. As well, movement of the carbon dioxide over horizontal distances is minimized, so as to reduce energy consumption. To this end, the photobioreactor 12 is, or are, relatively taller, and provide a reduced footprint, so as to increase carbon dioxide residence time while conserving energy.

In some embodiments, for example, a supplemental nutrient supply 42 is supplied to the reaction zone 10. In some embodiments, for example, the supplemental nutrient supply 42 is effected by a pump, such as a dosing pump. In other embodiments, for example, the supplemental nutrient supply 42 is supplied manually to the reaction zone 10. Nutrients within the reaction zone 10 are processed or consumed by the phototrophic biomass, and it is desirable, in some circumstances, to replenish the processed or consumed nutrients. A suitable nutrient composition is “Bold's Basal Medium”, and this is described in Bold, H. C. 1949, The morphology of Chlamydomonas chlamydogama sp. nov. Bull. Torrey Bot. Club. 76: 101-8 (see also Bischoff, H. W. and Bold, H. C. 1963, Phycological Studies IV. Some soil algae from Enchanted Rock and related algal species, Univ. Texas Publ. 6318: 1-95, and Stein J. (ED.) Handbook of Phycological Methods, Culture methods and growth measurements, Cambridge University Press, pp. 7-24). The supplemental nutrient supply 42 is supplied for supplementing the nutrients provided within the reaction zone, such as “Bold's Basal Medium”, or one or more dissolved components thereof. In this respect, in some embodiments, for example, the supplemental nutrient supply 42 includes “Bold's Basal Medium”. In some embodiments for example, the supplemental nutrient supply 42 includes one or more dissolved components of “Bold's Basal Medium”, such as NaNO₃, CaCl₂, MgSO₄, KH₂PO₄, NaCl, or other ones of its constituent dissolved components.

In some of these embodiments, the rate of supply of the supplemental nutrient supply 42 to the reaction zone 10 is controlled to align with a desired rate of growth of the phototrophic biomass in the reaction zone 10. In some embodiments, for example, regulation of nutrient addition is monitored by measuring any combination of pH, NO₃ concentration, and conductivity in the reaction zone 10.

In some embodiments, for example, a supply of the supplemental aqueous material supply 44 is effected to the reaction zone 10 so as to replenish water within the reaction zone 10 of the photobioreactor 12. In some embodiments, for example, and as further described below, the supplemental aqueous material supply 24 effects the discharge of product from the photobioreactor 12. For example, the supplemental aqueous material supply 24 effects the discharge of product from the photobioreactor 12 as an overflow.

In some embodiments, for example, the supplemental aqueous material is water. In some embodiments, for example, the supplemental aqueous material supply 44 includes at least one of: (a) aqueous material 70 that has been condensed from the reaction zone feed material 22 while the reaction zone feed material 22 is cooled before being supplied to the reaction zone 10, and (b) aqueous material that has been separated from a discharged phototrophic biomass-comprising product 58. In some embodiments, for example, the supplemental aqueous material supply 44 is derived from an independent source (i.e., a source other than the process), such as a municipal water supply.

In some embodiments, for example, the supplemental aqueous material supply 44 is supplied by the pump 281. In some of these embodiments, for example, the supplemental aqueous material supply 44 is continuously supplied to the reaction zone 10.

In some embodiments, for example, at least a fraction of the supplemental aqueous material supply 44 is supplied from a container 28, which is further described below. At least a fraction of aqueous material which is discharged from the process is recovered and supplied to the container 28 to provide supplemental aqueous material in the container 28.

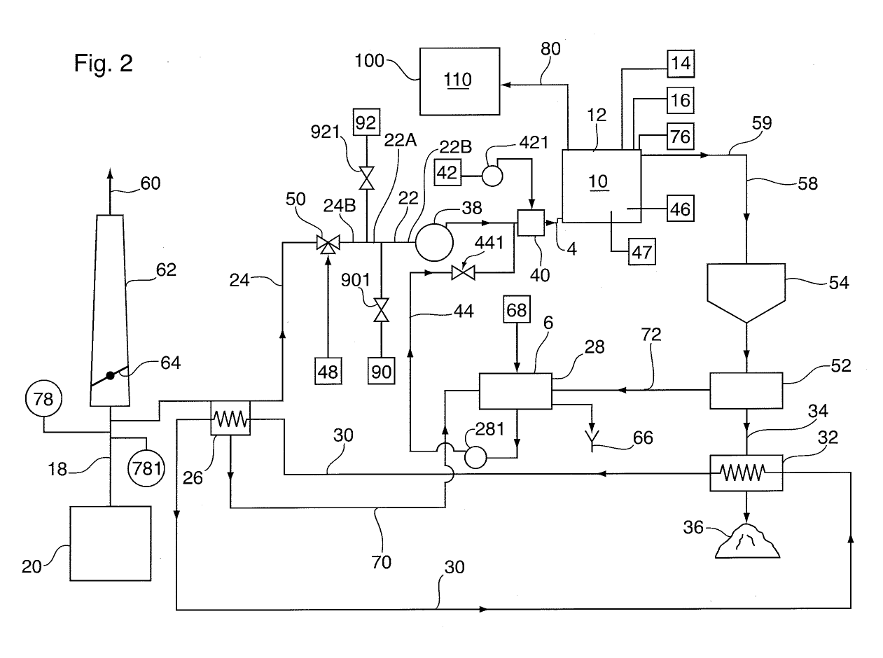

Referring to FIG. 2, in some embodiments, the supplemental nutrient supply 42 and the supplemental aqueous material supply 44 are supplied to the reaction zone feed material 22 through the sparger 40 before being supplied to the reaction zone 10. In those embodiments where the reaction zone 10 is disposed in the photobioreactor 12, in some of these embodiments, for example, the sparger 40 is disposed externally of the photobioreactor 12. In some embodiments, it is desirable to mix the reaction zone feed material 22 with the supplemental nutrient supply 42 and the supplemental aqueous material supply 44 within the sparger 40, as this effects better mixing of these components versus separate supplies of the reaction zone feed material 22, the supplemental nutrient supply 42, and the supplemental aqueous material supply 44. On the other hand, the rate of supply of the reaction zone feed material 22 to the reaction zone 10 is limited by virtue of saturation limits of gaseous material of the reaction zone feed material 22 in the combined mixture. Because of this trade-off, such embodiments are more suitable when response time required for providing a modulated supply of carbon dioxide to the reaction zone 10 is not relatively immediate, and this depends on the biological requirements of the phototrophic organisms being used.

In some embodiments, for example, at least a fraction of the supplemental nutrient supply 42 is mixed with the supplemental aqueous material in the container 28 to provide a nutrient-enriched supplemental aqueous material supply 44, and the nutrient-enriched supplemental aqueous material supply 44 is supplied directly to the reaction zone 10 or is mixed with the reaction zone feed material 22 in the sparger 40. In some embodiments, for example, the direct or indirect supply of the nutrient-enriched supplemental aqueous material supply is effected by a pump.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, at least one input to the reaction zone 10 is modulated based on the molar rate at which carbon dioxide is being supplied by the gaseous exhaust material producing process 20 to the reaction zone feed material 22. In some of these embodiments, the exposing of the phototrophic biomass disposed in the reaction zone 10 to photosynthetically active light radiation is effected while the modulating of at least one input is being effected.

As above-described, modulating of a input is any one of initiating, terminating, increasing, decreasing, or otherwise changing the input. An input to the reaction zone 10 is an input whose supply to the reaction zone 10 is material to the rate of growth of the phototrophic biomass within the reaction zone 10. Exemplary inputs to the reaction zone include transmission of an intensity of photosynthetically active light radiation of a characteristic intensity to the reaction zone 10, and supply of a molar rate of supply of supplemental nutrient supply 42 to the reaction zone 10.

In this respect, modulating the intensity of photosynthetically active light radiation being transmitted to the reaction zone is any one of: initiating supply of photosynthetically active light radiation being transmitted to the reaction zone, terminating supply of photosynthetically active light radiation being transmitted to the reaction zone, increasing the intensity of photosynthetically active light radiation being transmitted to the reaction zone, and decreasing the intensity of photosynthetically active light radiation being transmitted to the reaction zone. In some embodiments, for example, the modulating of the intensity of photosynthetically active light radiation being transmitted to the reaction zone includes modulating of the intensity of photosynthetically active light radiation to which at least a fraction of the carbon dioxide-enriched phototrophic biomass is exposed.

Modulating the molar rate of supply of supplemental nutrient supply 42 to the reaction zone is any one of initiating the supply of supplemental nutrient supply 42 to the reaction zone, terminating the supply of supplemental nutrient supply 42 to the reaction zone, increasing the molar rate of supply of supplemental nutrient supply 42 to the reaction zone, or decreasing the molar rate of supply of supplemental nutrient supply 42 to the reaction zone.

In some embodiments, for example, the gaseous exhaust material reaction zone supply 24 is supplied as a flow to the reaction zone feed material 22, and an indication of the molar rate of supply of carbon dioxide being supplied by the gaseous exhaust material producing process 20 (as the gaseous exhaust material reaction zone supply 24) to the reaction zone feed material 22 is the sensed molar flow rate of the gaseous exhaust material 18 being produced by the gaseous exhaust material producing process 20. In this respect, in some embodiments, for example, a flow sensor 78 is provided for sensing the molar flow rate of the gaseous exhaust material 18 being produced by the gaseous exhaust material producing process 20, and transmitting a signal representative of the molar flow rate of the gaseous exhaust material 18 to the controller. Upon the controller receiving a signal from the flow sensor 78 which is representative of the molar flow rate of the gaseous exhaust material 18, the controller effects modulation of at least one input to the reaction zone 10 based on the sensed molar flow rate of the gaseous exhaust material 18 being produced by the gaseous exhaust material producing process 20. In some embodiments, the modulation of at least one input includes effecting at least one of (a) initiation of, or an increase in the intensity of, photosynthetically active light radiation transmission to the reaction zone 10, and (b) initiation of, or an increase in the molar rate of supply of, a supplemental nutrient supply 42 to the reaction zone 10.

In some embodiments, for example, an indication of the molar rate of supply of carbon dioxide being supplied by the gaseous exhaust material producing process (as the gaseous exhaust material reaction zone supply 24) to the reaction zone feed material 22 is the sensed molar concentration of carbon dioxide of the gaseous effluent material 18 being produced by the gaseous exhaust material producing process 20. Because any of the discharged gaseous effluent material 18 that is supplied to the reaction zone feed material 22 is supplied as the gaseous exhaust material reaction zone supply 24, the sensing of the molar concentration of carbon dioxide of the discharged gaseous effluent material 18 includes sensing of the molar concentration of carbon dioxide of the gaseous exhaust material reaction zone supply 24. In this respect, in some embodiments, for example, a carbon dioxide sensor 781 is provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller receiving a signal from the carbon dioxide sensor 781 which is representative of a molar concentration of carbon dioxide of the gaseous exhaust material 18, the controller effects modulation of at least one input to the reaction zone 10 based on the sensed molar concentration of carbon dioxide of the gaseous exhaust material 18. In some embodiments, the modulation of at least one input includes effecting at least one of: (a) initiation of, or an increase in the intensity of, photosynthetically active light radiation transmission to the reaction zone 10, and (b) initiation of, an increase in the molar rate of supply of, a supplemental nutrient supply 42 to the reaction zone 10.

In some embodiments, for example, an indication of the molar rate of supply of carbon dioxide being supplied by the gaseous exhaust material producing process (as the gaseous exhaust material reaction zone supply 24) to the reaction zone feed material 22 is the combination of the sensed molar flow rate of the gaseous exhaust material 18 being produced by the gaseous exhaust material producing process 20 and the sensed molar concentration of carbon dioxide of the gaseous effluent material 18 being produced by the gaseous exhaust material producing process 20. The combination of the sensed molar flow rate of the gaseous exhaust material 18 being produced by the gaseous exhaust material producing process 20 and the sensed molar concentration of carbon dioxide of the gaseous effluent material 18 being produced by the gaseous exhaust material producing process 20 provides a sensed molar rate of supply of carbon dioxide, being supplied by the gaseous exhaust material producing process to the reaction zone feed material 22, that is representative of the (actual) molar rate of supply of carbon dioxide being supplied by the gaseous exhaust material producing process to the reaction zone feed material 22. In this respect, a flow sensor 78 is provided for sensing the molar flow rate of the gaseous exhaust material 18 being produced, and transmitting a signal representative of the molar flow rate of the gaseous exhaust material 18 to the controller. In this respect also, a carbon dioxide sensor 781 is provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller receiving a flow sensor signal from the flow sensor 78, which is representative of a molar flow rate of the gaseous exhaust material 18, and a carbon dioxide sensor signal from a carbon dioxide sensor 781, representative of a molar concentration of carbon dioxide of the gaseous exhaust material 18, and determining a sensed molar rate of supply of carbon dioxide, being supplied by the gaseous exhaust material producing process to the reaction zone feed material 22, that is representative of the molar rate of supply of carbon dioxide being supplied by the gaseous exhaust material producing process to the reaction zone feed material 22, based upon the received flow sensor signal and the received carbon dioxide sensor signal, the controller effects modulation of at least one input to the reaction zone 10 based on the sensed molar rate of supply of carbon dioxide being supplied by the gaseous exhaust material producing process to the reaction zone feed material 22. In some embodiments, the modulation of at least one input includes effecting at least one of: (a) initiation of, or an increase in the intensity of, photosynthetically active light radiation transmission to the reaction zone 10, and (b) initiation of, an increase in the molar rate of supply of, a supplemental nutrient supply 42 to the reaction zone 10.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, when an indication of a change in the molar rate of supply of carbon dioxide being supplied by the gaseous exhaust material producing process 20 to the reaction zone feed material 22 is sensed, modulation of at least one input to the reaction zone 10 is effected. In some of these embodiments, the exposing of the phototrophic biomass disposed in the reaction zone 10 to photosynthetically active light radiation is effected while the modulating of at least one input is being effected.

As above-described, modulating of an input is any one of initiating, terminating, increasing, or decreasing the input. Exemplary inputs to the reaction zone include transmission of an intensity of photosynthetically active light radiation of a characteristic intensity to the reaction zone 10, and supply of a molar rate of supply of supplemental nutrient supply 42 to the reaction zone 10.

As also above-described, modulating the intensity of photosynthetically active light radiation being transmitted to the reaction zone is any one of: initiating supply of photosynthetically active light radiation being transmitted to the reaction zone, terminating supply of photosynthetically active light radiation being transmitted to the reaction zone, increasing the intensity of photosynthetically active light radiation being transmitted to the reaction zone, and decreasing the intensity of photosynthetically active light radiation being transmitted to the reaction zone. In some embodiments, for example, the modulating of the intensity of photosynthetically active light radiation being transmitted to the reaction zone includes modulating of the intensity of photosynthetically active light radiation to which at least a fraction of the carbon dioxide-enriched phototrophic biomass is exposed.

As also above-described, modulating the molar rate of supply of supplemental nutrient supply 42 to the reaction zone is any one of initiating the supply of supplemental nutrient supply 42 to the reaction zone, terminating the supply of supplemental nutrient supply 42 to the reaction zone, increasing the molar rate of supply of supplemental nutrient supply 42 to the reaction zone, or decreasing the molar rate of supply of supplemental nutrient supply 42 to the reaction zone.

In some embodiments, for example, and as also above-described, the modulating of the intensity of the photosynthetically active light radiation is effected by a controller. In some embodiments, for example, to increase or decrease light intensity of a light source, the controller changes the power output to the light source from the power supply, and this can be effected by controlling either one of voltage or current. As well, in some embodiments, for example, the modulating of the molar rate of supply of the supplemental nutrient supply 42 is also effected by a controller. To modulate the molar rate of supply of the supplemental nutrient supply 42, the controller can control a dosing pump 421 to provide a desired flow rate of the supplemental nutrient supply 42.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, when an indication of an increase in the molar rate of supply of carbon dioxide being supplied by the gaseous exhaust material producing process 20 to the reaction zone feed material 22 is sensed, the modulating of at least one input includes: (i) initiating transmission of the photosynthetically active light radiation to the reaction zone 10, or (ii) effecting an increase in the intensity of the photosynthetically active light radiation being transmitted to the reaction zone 10. In some embodiments, for example, the increase in the intensity of the photosynthetically active light radiation is proportional to the increase in the molar rate of supply of carbon dioxide in the gaseous exhaust material reaction zone supply 24.

In some embodiments, for example, upon the initiating of the supply of photosynthetically active light radiation being transmitted to the reaction zone, or the increasing of the intensity of photosynthetically active light radiation being transmitted to the reaction zone, cooling of a light source, that is provided in the reaction zone 10 and that is supplying the photosynthetically active light radiation to the reaction zone, is effected. The cooling is effected for mitigating heating of the reaction zone by any thermal energy that is dissipated from the light source while the light source is supplying the photosynthetically active light radiation to the reaction zone. Heating of the reaction zone 10 increases the temperature of the reaction zone. In some embodiments, excessive temperature within the reaction zone 10 is deleterious to the phototrophic biomass. In some embodiments, for example, the light source is disposed in a liquid light guide and a heat transfer fluid is disposed within the liquid light guide, and the cooling is effected by increasing the rate of exchanges of the heat transfer fluid within the liquid light guide.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, when an indication of an increase in the molar rate of supply of carbon dioxide being supplied by the gaseous exhaust material producing process 20 to the reaction zone feed material 22 is sensed the modulating of at least one input includes: (i) initiating supply of the supplemental nutrient supply 42 to the reaction zone, or (ii) effecting an increase in the molar rate of supply of the supplemental nutrient supply 42 to the reaction zone 10.

In some embodiments, the gaseous exhaust material reaction zone supply 24 is supplied as a flow to the reaction zone feed material 22, and the indication of an increase in the molar rate of supply of carbon dioxide being supplied by the gaseous exhaust material producing process 20 (as the gaseous exhaust material reaction zone supply 24), to the reaction zone feed material 22, which is sensed is an increase in the sensed molar flow rate of the gaseous exhaust material 18 being produced by the gaseous exhaust material producing process 20. In this respect, in some embodiments, for example, a flow sensor 78 is provided for sensing the molar flow rate of the gaseous exhaust material 18 being produced, and transmitting a signal representative of the molar flow rate of the gaseous exhaust material 18 to the controller. Upon the controller comparing a received signal from the flow sensor 78 which is representative of a current molar flow rate of the gaseous exhaust material 18 to a previously received signal, and determining that an increase in the molar flow rate of the gaseous exhaust material 18 has been effected, the controller effects at least one of: (a) initiation of, or an increase in the intensity of, photosynthetically active light radiation transmission to the reaction zone 10, and (b) initiation of, or an increase in the molar rate of supply of, a supplemental nutrient supply 42 to the reaction zone 10.

In some embodiments, for example, the indication of an increase in the molar rate of supply of carbon dioxide being supplied by the gaseous exhaust producing process 20 (as the gaseous exhaust material reaction zone supply 24), to the reaction zone feed material 22, which is sensed is an increase in the sensed molar concentration of carbon dioxide of the discharged gaseous effluent material 18. Because any of the discharged gaseous effluent material 18 that is supplied to the reaction zone feed material 22 is supplied as the gaseous exhaust material reaction zone supply 24, the sensing of the molar concentration of carbon dioxide of the discharged gaseous effluent material 18 includes sensing of the molar concentration of carbon dioxide of the gaseous exhaust material reaction zone supply 24. In this respect, in some embodiments, for example, a carbon dioxide sensor 781 is provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller comparing a received signal from the carbon dioxide sensor 781 which is representative of a current molar concentration of carbon dioxide of the gaseous exhaust material 18 to a previously received signal, and determining that an increase in the molar concentration of carbon dioxide of the gaseous exhaust material 18 has been effected, the controller effects at least one of: (a) initiation of, or an increase in the intensity of, photosynthetically active light radiation transmission to the reaction zone 10, and (b) initiation of, or an increase in the molar rate of supply of, a supplemental nutrient supply 42 to the reaction zone 10.

In some embodiments, for example, the indication of an increase in the molar rate of supply of carbon dioxide being supplied by the gaseous exhaust producing process 20 (as the gaseous exhaust material reaction zone supply 24), to the reaction zone feed material 22, which is sensed is an increase in the sensed molar rate of supply of carbon dioxide, being supplied by the gaseous exhaust material producing process to the reaction zone feed material 22, that is representative of the (actual) molar rate of supply of carbon dioxide being supplied by the gaseous exhaust material producing process to the reaction zone feed material 22, and that is based on the combination of the sensed molar flow rate of the gaseous exhaust material 18 being produced by the gaseous exhaust material producing process 20 and the sensed molar concentration of carbon dioxide of the gaseous effluent material 18 being produced by the gaseous exhaust material producing process 20. In this respect, a flow sensor 78 is provided for sensing the molar flow rate of the gaseous exhaust material 18 being produced, and transmitting a signal representative of the molar flow rate of the gaseous exhaust material 18 to the controller. In this respect also, a carbon dioxide sensor 781 is provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller receiving a current flow sensor signal from the flow sensor 78, which is representative of a current molar flow rate of the gaseous exhaust material 18, and a current carbon dioxide sensor signal from a carbon dioxide sensor 781, representative of a current molar concentration of carbon dioxide of the gaseous exhaust material 18, and determining a current sensed molar rate of supply of carbon dioxide, being supplied by the gaseous exhaust material producing process to the reaction zone feed material 22, that is representative of the (actual) current molar rate of supply of carbon dioxide being supplied by the gaseous exhaust material producing process to the reaction zone feed material 22, based upon the received flow sensor signal and the received carbon dioxide sensor signal, and comparing the current sensed molar rate of supply of carbon dioxide to a previously sensed molar rate of supply of carbon dioxide that is based upon a previously received flow sensor signal and a previously received carbon dioxide sensor signal, and is representative of a previous molar rate of supply of carbon dioxide being supplied by the gaseous exhaust material producing process to the reaction zone feed material 22, and determining that an increase in the sensed molar rate of supply of carbon dioxide being supplied by the gaseous exhaust material producing process to the reaction zone feed material 22, the controller effects at least one of: (a) initiation of, or an increase in the intensity of, photosynthetically active light radiation transmission to the reaction zone 10, and (b) initiation of, or an increase in the molar rate of supply of, a supplemental nutrient supply 42 to the reaction zone 10.

In some embodiments, for example, any one of: (a) an increase in the sensed molar flow rate of the gaseous exhaust material 18 being produced, (b) an increase in the sensed molar concentration of carbon dioxide of the gaseous exhaust material 18 being produced, or (c) an increase in the sensed molar rate of supply of carbon dioxide being supplied by the gaseous exhaust material producing process to the reaction zone feed material 22, is an indicator of an impending increase in the molar rate of supply of carbon dioxide to the reaction zone feed material 22. Because an increase in the rate of molar supply of carbon dioxide to the reaction zone feed material 22 is impending, the molar rate of supply of at least one condition for growth (i.e., increased rate of supply of carbon dioxide) of the phototrophic biomass is increased, and the rates of supply of other inputs, relevant to such growth, are correspondingly initiated or increased, in anticipation of growth of the phototrophic biomass in the reaction zone 10.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, when an indication of a decrease in the molar rate of supply of carbon dioxide being supplied by the gaseous exhaust material producing process 20 to the reaction zone feed material 22 is sensed, the modulating of at least one input includes effecting at least one of: (i) terminating transmission of the photosynthetically active light radiation to the reaction zone 10, or (ii) effecting a decrease in the intensity of the photosynthetically active light radiation being transmitted to the reaction zone 10. In some embodiments, for example, the increase in the intensity of the photosynthetically active light radiation is proportional to the increase in the molar rate of supply of carbon dioxide in the gaseous exhaust material reaction zone supply 24.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, when an indication of a decrease in the molar rate of supply of carbon dioxide being supplied by the gaseous exhaust material producing process 20 to the reaction zone feed material 22 is sensed, the modulating of at least one input includes effecting at least one of: (i) terminating supply of the supplemental nutrient supply 42 to the reaction zone, or (ii) effecting a decrease in the molar rate of supply of the supplemental nutrient supply 42 to the reaction zone 10.

In some embodiments, for example, the gaseous exhaust material reaction zone supply 24 is supplied as a flow to the reaction zone feed material 22, and the indication of a decrease in the molar rate of supply of carbon dioxide being supplied by the gaseous exhaust material producing process 20 (as the gaseous exhaust material reaction zone supply 24), to the reaction zone feed material 22, which is sensed is a decrease in the molar flow rate of the gaseous exhaust material 18 being produced by the gaseous exhaust material producing process 20. In this respect, in some embodiments, for example, a flow sensor 78 is provided for sensing the molar flow rate of the gaseous exhaust material 18 being produced, and transmitting a signal representative of the molar flow rate of the gaseous exhaust material 18 to the controller. Upon the controller comparing a received signal from the flow sensor 78 which is representative of a current molar flow rate of the gaseous exhaust material 18 to a previously received signal, and determining that a decrease in the molar flow rate of the gaseous exhaust material 18 has been effected, the controller effects at least one of: (a) a decrease in the intensity of, or termination of, the photosynthetically active light radiation transmission to the reaction zone 10, and (b) a decrease in the molar rate of supply of, or termination of supply of, of a supplemental nutrient supply 42 to the reaction zone 10.

In some embodiments, for example, the indication of a decrease in the molar rate of supply of carbon dioxide being supplied by the gaseous exhaust producing process 20 (as the gaseous exhaust material reaction zone supply 24), to the reaction zone feed material 22, which is sensed is a decrease in the molar concentration of carbon dioxide of the discharged gaseous effluent material 18. Because any of the discharged gaseous effluent material 18 that is supplied to the reaction zone feed material 22 is supplied as the gaseous exhaust material reaction zone supply 24, the sensing of the molar concentration of carbon dioxide of the discharged gaseous effluent material 18 includes sensing of the molar concentration of carbon dioxide of the gaseous exhaust material reaction zone supply 24. In this respect, in some embodiments, for example, a carbon dioxide sensor 781 is provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller comparing a received signal from the carbon dioxide sensor 781 which is representative of a current molar concentration of carbon dioxide of the gaseous exhaust material 18 to a previously received signal, and determining that a decrease in the molar concentration of carbon dioxide of the gaseous exhaust material 18 has been effected, the controller effects at least one of: (a) a decrease in the intensity of, or termination of, the photosynthetically active light radiation transmission to the reaction zone 10, and (b) a decrease in the molar rate of supply of, or termination of supply of, of a supplemental nutrient supply 42 to the reaction zone 10.

In some embodiments, for example, the indication of a decrease in the molar rate of supply of carbon dioxide being supplied by the gaseous exhaust producing process 20 (as the gaseous exhaust material reaction zone supply 24), to the reaction zone feed material 22, which is sensed is a decrease in the sensed molar rate of supply of carbon dioxide, being supplied by the gaseous exhaust material producing process to the reaction zone feed material 22, that is representative of the (actual) molar rate of supply of carbon dioxide being supplied by the gaseous exhaust material producing process to the reaction zone feed material 22, and that is based on the combination of the sensed molar flow rate of the gaseous exhaust material 18 being produced by the gaseous exhaust material producing process 20 and the sensed molar concentration of carbon dioxide of the gaseous effluent material 18 being produced by the gaseous exhaust material producing process 20. In this respect, a flow sensor 78 is provided for sensing the molar flow rate of the gaseous exhaust material 18 being produced, and transmitting a signal representative of the molar flow rate of the gaseous exhaust material 18 to the controller. In this respect also, a carbon dioxide sensor 781 is provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller receiving a current flow sensor signal from the flow sensor 78, which is representative of a current molar flow rate of the gaseous exhaust material 18, and a current carbon dioxide sensor signal from a carbon dioxide sensor 781, representative of a current molar concentration of carbon dioxide of the gaseous exhaust material 18, and determining a current sensed molar rate of supply of carbon dioxide, being supplied by the gaseous exhaust material producing process to the reaction zone feed material 22, that is representative of the (actual) current molar rate of supply of carbon dioxide being supplied by the gaseous exhaust material producing process to the reaction zone feed material 22, based upon the received flow sensor signal and the received carbon dioxide sensor signal, and comparing the current sensed molar rate of supply of carbon dioxide to a previously sensed molar rate of supply of carbon dioxide that is based upon a previously received flow sensor signal and a previously received carbon dioxide sensor signal, and is representative of a previous molar rate of supply of carbon dioxide being supplied by the gaseous exhaust material producing process to the reaction zone feed material 22, and determining that a decrease in the sensed molar rate of supply of carbon dioxide being supplied by the gaseous exhaust material producing process to the reaction zone feed material 22, the controller effects at least one of: (a) a decrease in the intensity of, or termination of, the photosynthetically active light radiation transmission to the reaction zone 10, and (b) a decrease in the molar rate of supply of, or termination of supply of, of a supplemental nutrient supply 42 to the reaction zone 10.

In some embodiments, for example, any one of: (a) a decrease in the molar flow rate of the gaseous exhaust material 18 being produced, (b) a decrease in the molar concentration of carbon dioxide of the gaseous exhaust material 18 being produced, or (c) a decrease in the sensed molar rate of supply of carbon dioxide being supplied by the gaseous exhaust material producing process to the reaction zone feed material 22, is an indicator of an impending decrease in the rate of molar supply of carbon dioxide to the reaction zone feed material 22. Because a decrease in the rate of molar supply of carbon dioxide to reaction zone feed material 22 is impending, the rate of supply of other inputs, which would otherwise be relevant to phototrophic biomass growth, are correspondingly reduced or terminated to conserve such inputs.

In some embodiments, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, when an indication of a decrease in the molar rate of supply of carbon dioxide being supplied by the gaseous exhaust material producing process 20, to the reaction zone feed material 22, is sensed, either the molar rate of supply of a supplemental carbon dioxide supply 92 to the reaction zone feed material 22 is increased, or supply of the supplemental carbon dioxide supply 92 to the reaction zone feed material 22 is initiated. In some embodiments, for example, the source of the supplemental carbon dioxide supply 92 is a carbon dioxide cylinder. In some embodiments, for example, the source of the supplemental carbon dioxide supply 92 is a supply of air. In some of these embodiments, the exposing of the phototrophic biomass disposed in the reaction zone 10 to photosynthetically active light radiation is effected while the increasing of the molar rate of supply, or the initiation of supply, of the supplemental carbon dioxide supply 92 to the reaction zone feed material 22 is being effected. In some embodiments, for example, the indication of a decrease in the molar rate of supply of carbon dioxide (being supplied by the supply 24) is any of the indications described above. In some embodiments, for example, the supplemental carbon dioxide supply 92 is provided for compensating for the decrease in the molar rate of supply of carbon dioxide being supplied by the gaseous exhaust material producing process 20 to the reaction zone feed material 22, with a view to sustaining a constant growth rate of the phototrophic biomass, when it is believed that the decrease is only of a temporary nature (such as less than two weeks).

In those embodiments where the increasing of the molar rate of supply, or the initiation of supply, of a supplemental carbon dioxide supply 92 to the reaction zone 10 is effected in response to the sensing of an indication of a decrease in the molar rate of supply of carbon dioxide being supplied to the reaction zone feed material 22 by the gaseous exhaust material producing process 20 as gaseous exhaust material reaction zone supply 24, when the gaseous exhaust material reaction zone supply 24 is supplied as a flow to the reaction zone feed material 22, and the indication of a decrease in the molar rate of supply of carbon dioxide being supplied by the gaseous exhaust material producing process 20 (as the gaseous exhaust material reaction zone supply 24), to the reaction zone feed material 22, which is sensed is a decrease in the molar flow rate of the gaseous exhaust material 18 being produced by the gaseous exhaust material producing process 20, in some of these embodiments, for example, a flow sensor 78 is provided for sensing the molar flow rate of the gaseous exhaust material 18 being produced, and transmitting a signal representative of the molar flow rate of the gaseous exhaust material 18 to the controller. Upon the controller comparing a received signal from the flow sensor 78 which is representative of a current molar flow rate of the gaseous exhaust material 18 to a previously received signal, and determining that a decrease in the molar flow rate of the gaseous exhaust material 18 has been effected, the controller actuates the opening of a flow control element, such as a valve 921, to initiate supply of the supplemental carbon dioxide supply 92 to the reaction zone feed material 22, or to effect increasing of the molar rate of supply of the supplemental carbon dioxide supply to the reaction zone feed material 22.

In those embodiments where the increasing of the molar rate of supply, or the initiation of supply, of a supplemental carbon dioxide supply 92 to the reaction zone 10 is effected in response to the sensing of an indication of a decrease in the molar rate of supply of carbon dioxide being supplied to the reaction zone feed material 22 by the gaseous exhaust material producing process 20 as gaseous exhaust material reaction zone supply 24, when the indication of a decrease in the molar rate of supply of carbon dioxide being supplied by the gaseous exhaust material producing process 20 (as the gaseous exhaust material reaction zone supply 24), to the reaction zone feed material 22, which is sensed is a decrease in the molar concentration of carbon dioxide of the gaseous exhaust material 18 being produced by the gaseous exhaust material producing process 20 (or the molar concentration of carbon dioxide of the gaseous exhaust material reaction zone supply 24), in some embodiments, for example, a carbon dioxide sensor 781 is provided for sensing the molar concentration of carbon dioxide of the gaseous exhaust material 18 being produced (or the molar concentration of carbon dioxide the gaseous exhaust material reaction zone supply 24), and transmitting a signal representative of the molar concentration of carbon dioxide of the gaseous exhaust material 18 being produced (or of the molar concentration of carbon dioxide of the gaseous exhaust material reaction zone supply 24) to the controller. Upon the controller comparing a received signal from the carbon dioxide sensor 781 which is representative of a current molar concentration of carbon dioxide of the gaseous exhaust material 18 (or representative of a current molar concentration of carbon dioxide of the gaseous exhaust material reaction zone supply 24) to a previously received signal, and determining that a decrease in the molar concentration of carbon dioxide of the gaseous exhaust material 18 has been effected, the controller actuates the opening of a flow control element, such as a valve 921, to initiate supply of the supplemental carbon dioxide supply 92 to the reaction zone feed material 22, or to effect increasing of the molar rate of supply of the supplemental carbon dioxide supply to the reaction zone feed material 22.

In those embodiments where the increasing of the molar rate of supply, or the initiation of supply, of a supplemental carbon dioxide supply 92 to the reaction zone 10 is effected in response to the sensing of an indication of a decrease in the molar rate of supply of carbon dioxide being supplied to the reaction zone feed material 22 by the gaseous exhaust material producing process 20 as gaseous exhaust material reaction zone supply 24, when the indication of a decrease in the molar rate of supply of carbon dioxide being supplied by the gaseous exhaust material producing process 20 (as the gaseous exhaust material reaction zone supply 24), to the reaction zone feed material 22, which is sensed is a decrease in the sensed molar rate of supply of carbon dioxide, being supplied by the gaseous exhaust material producing process to the reaction zone feed material 22, that is representative of the (actual) molar rate of supply of carbon dioxide being supplied by the gaseous exhaust material producing process to the reaction zone feed material 22, and that is based on the combination of the sensed molar flow rate of the gaseous exhaust material 18 being produced by the gaseous exhaust material producing process 20 and the sensed molar concentration of carbon dioxide of the gaseous effluent material 18 being produced by the gaseous exhaust material producing process 20, a flow sensor 78 is provided for sensing the molar flow rate of the gaseous exhaust material 18 being produced, and transmitting a signal representative of the molar flow rate of the gaseous exhaust material 18 to the controller, and a carbon dioxide sensor 781 is also provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller receiving a current flow sensor signal from the flow sensor 78, which is representative of a current molar flow rate of the gaseous exhaust material 18, and a current carbon dioxide sensor signal from a carbon dioxide sensor 781, representative of a current molar concentration of carbon dioxide of the gaseous exhaust material 18, and determining a current sensed molar rate of supply of carbon dioxide, being supplied by the gaseous exhaust material producing process to the reaction zone feed material 22, that is representative of the (actual) current molar rate of supply of carbon dioxide being supplied by the gaseous exhaust material producing process to the reaction zone feed material 22, based upon the received flow sensor signal and the received carbon dioxide sensor signal, and comparing the current sensed molar rate of supply of carbon dioxide to a previously sensed molar rate of supply of carbon dioxide that is based upon a previously received flow sensor signal and a previously received carbon dioxide sensor signal, and is representative of a previous molar rate of supply of carbon dioxide being supplied by the gaseous exhaust material producing process to the reaction zone feed material 22, and determining that a decrease in the sensed molar rate of supply of carbon dioxide being supplied by the gaseous exhaust material producing process to the reaction zone feed material 22, the controller actuates the opening of a flow control element, such as a valve 921, to initiate supply of the supplemental carbon dioxide supply 92 to the reaction zone feed material 22, or to effect increasing of the molar rate of supply of the supplemental carbon dioxide supply to the reaction zone feed material 22.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, a discharge of the gaseous exhaust material 18 from the gaseous exhaust material producing process 20 is modulated based on sensing of at least one carbon dioxide processing capacity indicator. In some embodiments, for example, the sensing of at least one of the at least one carbon dioxide processing capacity indicator is effected in the reaction zone 10. The modulating of the discharge of the gaseous exhaust material 18 includes modulating of a supply of the discharged gaseous exhaust material 18 supplied to the reaction zone feed material 22. As described above, any discharged gaseous exhaust material 18 that is supplied to the reaction zone feed material 22 is supplied as the gaseous exhaust material reaction zone supply 24. The gaseous exhaust material reaction zone supply 24 includes carbon dioxide. In some embodiments, for example, the discharged gaseous exhaust material 18 is provided in the form of a gaseous flow. In some embodiments, for example, the gaseous exhaust material reaction zone supply 24 is provided in the form of a gaseous flow. In some embodiments, for example, the exposing of the phototrophic biomass disposed in the reaction zone 10 to photosynthetically active light radiation is effected while the modulating of the discharge of the produced gaseous exhaust material 18 is being effected.

When the discharge of the gaseous exhaust material 18 from the gaseous exhaust material producing process 20 is modulated based on sensing of at least one carbon dioxide processing capacity indicator, in some embodiments, for example, the process further includes modulating of a supply of the discharged gaseous exhaust material 18 to another unit operation. The supply of the discharged gaseous exhaust material 18 to another unit operation defines a bypass gaseous exhaust material 60. The bypass gaseous exhaust material 60 includes carbon dioxide. The another unit operation converts the bypass gaseous exhaust material 60 such that its environmental impact is reduced.

The carbon dioxide processing capacity indicator which is sensed is any characteristic of the process that is suggestive of the capacity of the reaction zone 10 for receiving carbon dioxide and converting at least a fraction of the received carbon dioxide through photosynthesis effected by phototrophic biomass disposed within the reaction zone.

In some embodiments, for example, the carbon dioxide processing capacity indicator which is sensed is any characteristic of the process that is suggestive of the capacity of the reaction zone for receiving carbon dioxide and converting at least a fraction of the received carbon dioxide through photosynthesis effected by phototrophic biomass disposed within the reaction zone 10, such that the photosynthesis effects a desired growth rate of the phototrophic biomass within the reaction zone 10. In this respect, the sensing of the carbon dioxide processing capacity indicator is material to determining whether modulation of the discharge of the gaseous exhaust material 18 is required to effect a desired rate of growth of the phototrophic biomass within the reaction zone 10.

In some embodiments, for example, the carbon dioxide processing capacity indicator which is sensed is any characteristic of the process that is suggestive of the capacity of the reaction zone for receiving carbon dioxide and converting at least a fraction of the received carbon dioxide through photosynthesis effected by the phototrophic biomass disposed within the reaction zone 10, such that any discharge of carbon dioxide from the reaction zone is effected below an acceptable molar rate. In this respect, the sensing of the carbon dioxide processing capacity indicator is material to determining whether modulation of the discharge of the gaseous exhaust material 18 is required to effect an acceptable molar rate of discharge of the carbon dioxide from the reaction zone 10.

In some embodiments, for example, the carbon dioxide processing capacity indicator which is sensed is a pH within the reaction zone 10. In some embodiments, for example, the carbon dioxide processing capacity indicator which is sensed is a phototrophic biomass molar concentration within the reaction zone 10. Because any of phototrophic biomass-comprising product 58 that is being discharged from the reaction zone 10 includes a portion of material from within the reaction zone 10 (i.e., phototrophic biomass-comprising product 58 that is being discharged from the reaction zone 10 is supplied with material from within the reaction zone 10), the sensing of a carbon dioxide processing capacity indicator (such as the pH within the reaction zone, or the phototrophic biomass molar concentration within the reaction zone) includes sensing of the carbon dioxide processing capacity indicator within the phototrophic biomass-comprising product 58 that is being discharged from the reaction zone 10.

In some embodiments for example, the modulating of the supply of the discharge of the gaseous exhaust material 18 is based on sensing of two or more carbon dioxide processing capacity indicators within the reaction zone 10.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and when a carbon dioxide processing capacity indicator is sensed in the reaction zone 10 which is suggestive of a capacity of the reaction zone 10 for receiving an increased molar rate of supply of carbon dioxide, the modulating of the discharge of the gaseous exhaust material 18 includes initiating the supply of, or increasing the molar rate of supply of, the gaseous exhaust material reaction zone supply 24 to the reaction zone feed material 22 In those embodiments where the outlet of the gaseous exhaust material producing process 20 is co-operatively disposed with another unit operation to effect supply of the bypass gaseous exhaust material 60 to the another unit operation, and while the bypass gaseous exhaust material 60 is being supplied to the another unit operation, the modulating of the discharge of the gaseous exhaust material 18 further includes effecting a decrease to the molar rate of supply of, or terminating the supply of, the bypass gaseous exhaust material 60 to the another unit operation.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and when a carbon dioxide processing capacity indicator is sensed in the reaction zone 10 which is suggestive of a capacity of the reaction zone 10 for receiving a decreased molar rate of supply of carbon dioxide, the modulating of the discharge of the gaseous exhaust material 18 includes reducing the molar rate of supply of, or terminating the supply of, the gaseous exhaust material reaction zone supply 24 to the reaction zone feed material 22. In those embodiments where the outlet of the gaseous exhaust material producing process 20 is co-operatively disposed with another unit operation to effect supply of the bypass gaseous exhaust material 60 to the another unit operation, the modulating of the discharge of the gaseous exhaust material 18 further includes initiating the supply of, or effecting an increase to the molar rate of supply of, the bypass gaseous exhaust material 60 to the another unit operation.

In some embodiments, for example, the carbon dioxide processing capacity indicator is a pH within the reaction zone 10. Operating with a pH in the reaction zone 10 which is above the predetermined high pH (indicating an insufficient molar rate of supply of carbon dioxide to the reaction zone feed material 22), or which is below the predetermined low pH (indicating an excessive molar rate of supply of carbon dioxide to the reaction zone feed material 22), effects less than a desired growth rate of the phototrophic biomass, and, in the extreme, could effect death of the phototrophic biomass.

In this respect, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and when a pH is sensed in the reaction zone 10 that is above a predetermined high pH value, the modulating of the discharge of the gaseous exhaust material 18 includes initiating the supply of, or increasing the molar rate of supply of, the gaseous exhaust material reaction zone supply 24 to the reaction zone feed material 22. In those embodiments where the outlet of the gaseous exhaust material producing process 20 is co-operatively disposed with another unit operation to effect supply of the bypass gaseous exhaust material 60 to the another unit operation, and while the bypass gaseous exhaust material 60 is being supplied to the another unit operation, the modulating of the discharge of the gaseous exhaust material 18 further includes effecting a decrease to the molar rate of supply of, or terminating the supply of, the bypass gaseous exhaust material 60 to the another unit operation.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and when a pH is sensed in the reaction zone 10 that is below a predetermined low pH value, the modulating of the discharge of the gaseous exhaust material 18 includes reducing the molar rate of supply of, or terminating the supply of, the gaseous exhaust material reaction zone supply 24 to the reaction zone feed material 22. In those embodiments where the outlet of the gaseous exhaust material producing process 20 is co-operatively disposed with another unit operation to effect supply of the bypass gaseous exhaust material 60 to the another unit operation, the modulating of the discharge of the gaseous exhaust material 18 further includes initiating the supply of, or effecting an increase to the molar rate of supply of, the bypass gaseous exhaust material 60 to the another unit operation.

In some embodiments, for example, the pH which is sensed in the reaction zone is sensed in the reaction zone 10 with a pH sensor 46. The pH sensor 46 is provided for sensing the pH within the reaction zone, and transmitting a signal representative of the pH within the reaction zone to the controller.

In some embodiments, for example, the pH within the reaction zone is below a predetermined low pH value. In these circumstances, upon the controller comparing a received signal from the pH sensor 46 which is representative of the pH within the reaction zone 10 to a target value (i.e., the predetermined low pH value), and determining that the pH within the reaction zone 10 is below the predetermined low pH value, the controller responds by effecting reduction of the molar rate of supply of, or effecting termination of the supply of, the gaseous exhaust material reaction zone supply 24 to the reaction zone feed material 22. In some embodiments, for example, this is effected by actuating a flow control element 50 (such as a valve) with the controller. The flow control element 50 is provided and configured to selectively control the molar rate of flow of the gaseous exhaust material reaction zone supply 24 by selectively interfering with the flow of the gaseous exhaust material reaction zone supply 24, which is supplying the reaction zone feed material 22, by effecting pressure losses to the flow of the gaseous exhaust material reaction zone supply 24. In this respect, the reducing of the molar rate of supply, or the termination of the supply, of the gaseous exhaust material reaction zone supply 24 to the reaction zone feed material 22 is effected by the flow control element 50. The predetermined low pH value depends on the phototrophic organisms of the biomass. In some embodiments, for example, the predetermined low pH value can be as low as 4.0.

In those embodiments where the outlet of the gaseous exhaust material producing process 20 is co-operatively disposed with another unit operation to effect supply of the bypass gaseous exhaust material 60 to the another unit operation, in some of these embodiments, for example, upon the controller determining that the pH within the reaction zone 10 is below the predetermined low pH value, the controller further responds by effecting initiation of the supply of, or effecting an increase to the molar rate of supply of, the bypass gaseous exhaust material 60 to the another unit operation. In some embodiments, for example, the initiation of the supply, or the increase to the molar rate of supply of, the bypass gaseous exhaust material 60 to the another unit operation is effected by the controller by actuation of a valve disposed between the gaseous exhaust material producing process 20 and the another unit operation.

Also in those embodiments where the outlet of the gaseous exhaust material producing process 20 is co-operatively disposed with another unit operation to effect supply of the bypass gaseous exhaust material 60 to the another unit operation, in other ones of these embodiments, for example, the initiation of the supply, or the increase to the molar rate of supply of, the bypass gaseous exhaust material 60 to the another unit operation is effected when the pressure of the gaseous exhaust material 18 is above a predetermined set point pressure, wherein an increase in pressure of the gaseous exhaust material 18 to above the predetermined set point pressure is effected in response to a decrease of the molar rate of supply of, or the termination of the supply of, the gaseous exhaust material reaction zone supply 24 to the reaction zone feed material 22 which is effected by the controller in response to the determination that the sensed pH within the reaction zone is below a predetermined low pH value. In such embodiments, upon the controller determining that the sensed pH within the reaction zone by the pH sensor 47 is below a predetermined low pH value, the controller effects a decrease of the molar rate of supply of, or the termination of the supply of, the gaseous exhaust material reaction zone supply 24 to the reaction zone feed material 22, as described above. The decrease of the molar rate of supply of, or the termination of the supply of, the gaseous exhaust material reaction zone supply 24 to the reaction zone feed material 22 effects a corresponding increase in pressure upstream of the flow control element 50 such that the pressure of the gaseous exhaust material 18 becomes disposed above the predetermined set point pressure. When the pressure of the gaseous exhaust material 18 is above the predetermined set point pressure, the forces biasing closure of a closure element 64 (such as a valve), disposed between the gaseous exhaust material producing process 20 and the another unit operation, are exceeded by the fluid pressure forces acting to open the closure element 64, and there is effected an initiation of the opening of, or an increase to the opening of, the closure element 64. This initiation of the opening of, or the increase to the opening of, the closure element 64, effects the initiation of the supply of, or the increase to the molar rate of supply of, the bypass gaseous exhaust material 60 to the another unit operation.

In some embodiments, for example, the pH within the reaction zone is above a predetermined high pH value. In these circumstances, upon the controller comparing a received signal from the pH sensor 47 which is representative of the pH within the reaction zone 10 to a target value (i.e., the predetermined high pH value), and determining that the pH within the reaction zone 10 is above the predetermined high pH value, the controller responds by effecting initiation of the supply of, or effecting an increase to the molar rate of supply of, the gaseous exhaust material reaction zone supply 24 to the reaction zone feed material 22. In some embodiments, for example, this is effected by effecting initiation of supply of, or effecting an increase to the molar supply rate of, the gaseous exhaust material reaction zone supply 24 being supplied to the reaction zone feed material 22, such as by actuating the flow control element 50 with the controller. The predetermined high pH value depends on the phototrophic organisms of the biomass. In some embodiments, for example, the predetermined high pH value can be as high as 10.

In those embodiments where the outlet of the gaseous exhaust material producing process 20 is co-operatively disposed with another unit operation to effect supply of the bypass gaseous exhaust material 60 to the another unit operation, and while the bypass gaseous exhaust material 60 is being supplied to the another unit operation, in some of these embodiments, for example, upon the controller determining that the pH within the reaction zone 10 is above the predetermined high pH value, the controller further responds by effecting a decrease to the molar rate of supply of, or by effecting termination of the supply of, the bypass gaseous exhaust material 60 to the another unit operation. In some embodiments, for example, the decrease to the molar rate of supply of, or the termination of the supply of, the bypass gaseous exhaust material 60 to the another unit operation is effected by the controller by actuation of a valve disposed between the gaseous exhaust material producing process 20 and the another unit operation.

Also in those embodiments where the outlet of the gaseous exhaust material producing process 20 is co-operatively disposed with another unit operation to effect supply of the bypass gaseous exhaust material 60 to the another unit operation, and while the bypass gaseous exhaust material 60 is being supplied to the another unit operation, in other ones of these embodiments, for example, the decrease to the molar rate of supply of, or the termination of the supply of, the bypass gaseous exhaust material 60 to the another unit operation is effected when the pressure of the gaseous exhaust material 18 is below a predetermined set point pressure, wherein the decrease in pressure of the gaseous exhaust material 18 to below the predetermined set point pressure is effected in response to an initiation of the supply of, or an increase to the molar rate of supply of, the gaseous exhaust material reaction zone supply 24 to the reaction zone feed material 22, which is effected by the controller in response to the determination that the sensed pH within the reaction zone is above a predetermined high pH value. In such embodiments, upon the controller determining that the sensed pH within the reaction zone by the pH sensor 46 is above a predetermined high pH value, the controller effects initiation of the supply of, or effects an increase to the molar rate of supply of, the gaseous exhaust material reaction zone supply 24 to the reaction zone feed material 22, as described above. The initiation of the supply of, or the increase to the molar rate of supply of, the gaseous exhaust material reaction zone supply 24 to the reaction zone feed material 22 effects a corresponding decrease in pressure upstream of the flow control element 50 such that the pressure of the gaseous exhaust material 18 becomes disposed below the predetermined set point pressure. When the pressure of the gaseous exhaust material is below the predetermined minimum pressure, the forces biasing closure of a closure element 64 (such as a valve), disposed between the gaseous exhaust material producing process 20 and the another unit operation, exceed the fluid pressure forces acting to open the closure element 64, and there is effected a decrease in the opening of, or a closure of, the closure element 64. This decrease in the opening of, or the closure of, the closure element 64, effects the decrease to the molar rate of supply of, or the termination of the supply of, the bypass gaseous exhaust material 60 to the another unit operation.

In some embodiments, for example, the carbon dioxide processing capacity indicator is a phototrophic biomass concentration within the reaction zone 10. The phototrophic biomass concentration within the reaction zone In some embodiments, for example, it is desirable to control the concentration of the phototrophic biomass within the reaction zone 10, as, for example, higher overall yield of the harvested phototrophic biomass is effected when the concentration of the phototrophic biomass within the reaction zone 10 is maintained at a predetermined concentration or within a predetermined concentration range.

In this respect, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and when a phototrophic biomass concentration is sensed in the reaction zone 10 that is above a predetermined high phototrophic biomass concentration value, the modulating of the discharge of the gaseous exhaust material 18 includes reducing the molar rate of supply of, or terminating the supply of, the gaseous exhaust material reaction zone supply 24 to the reaction zone feed material 22. In those embodiments where the outlet of the gaseous exhaust material producing process 20 is co-operatively disposed with another unit operation to effect supply of the bypass gaseous exhaust material 60 to the another unit operation, the modulating of the discharge of the gaseous exhaust material 18 further includes initiating the supply of, or effecting an increase to the molar rate of supply of, the bypass gaseous exhaust material 60 to the another unit operation.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and when a phototrophic biomass concentration is sensed in the reaction zone 10 that is below a predetermined low phototrophic biomass concentration value, the modulating of the discharge of the gaseous exhaust material 18 includes initiating the supply of, or increasing the molar rate of supply of, the gaseous exhaust material reaction zone supply 24 to the reaction zone feed material 22. In those embodiments where the outlet of the gaseous exhaust material producing process 20 is co-operatively disposed with another unit operation to effect supply of the bypass gaseous exhaust material 60 to the another unit operation, and while the bypass gaseous exhaust material 60 is being supplied to the another unit operation, the modulating of the discharge of the gaseous exhaust material 18 further includes effecting a decrease to the molar rate of supply of, or terminating the supply of, the bypass gaseous exhaust material 60 to the another unit operation.

In some embodiments, the sensing of the phototrophic biomass concentration in the reaction zone 10 is effected with a cell counter 47. For example, a suitable cell counter is an AS-16F Single Channel Absorption Probe supplied by optek-Danulat, Inc. of Germantown, Wis., U.S.A. Other suitable devices for sensing phototrophic biomass concentration include other light scattering sensors, such as a spectrophotometer. As well, the phototrophic biomass concentration can be sensed manually, and then input manually into the controller for effecting the desired response.

In some embodiments, for example, the phototrophic biomass concentration within the reaction zone is below a predetermined low phototrophic biomass concentration value. In these circumstances, upon the controller comparing a received signal from the cell counter 47, which is representative of the phototrophic biomass concentration within the reaction zone 10, to a target value (i.e., the predetermined low phototrophic biomass concentration value), and determining that the phototrophic biomass concentration within the reaction zone 10 is below the low phototrophic biomass concentration value, the controller responds by effecting initiation of the supply of, or effecting an increase to the molar rate of supply of, the gaseous exhaust material reaction zone supply 24 to the reaction zone feed material 22. In some embodiments, for example, this is effected by actuating the flow control element 50 with the controller.

In those embodiments where the outlet of the gaseous exhaust material producing process 20 is co-operatively disposed with another unit operation to effect supply of the bypass gaseous exhaust material 60 to the another unit operation, and while the bypass gaseous exhaust material 60 is being supplied to the another unit operation, in some of these embodiments, for example, upon the controller comparing a received signal from the cell counter 47, which is representative of the phototrophic biomass concentration within the reaction zone 10, to the predetermined low phototrophic biomass concentration value, and determining that the phototrophic biomass concentration within the reaction zone 10 is below the low phototrophic biomass concentration value, the controller further responds by effecting a decrease to the molar rate of supply of, or by effecting the termination of the supply of, the bypass gaseous exhaust material 60 to the another unit operation. In some embodiments, for example, the decrease to the molar rate of supply of, or the termination of the supply of, the bypass gaseous exhaust material 60 to the another unit operation is effected by the controller by actuation of a valve disposed between the gaseous exhaust material producing process 20 and the another unit operation.

Also in those embodiments where the outlet of the gaseous exhaust material producing process 20 is co-operatively disposed with another unit operation to effect supply of bypass gaseous exhaust material 60 to the another unit operation, and while bypass gaseous exhaust material 60 is being supplied to the another unit operation, in other ones of these embodiments, for example, the decrease to the molar rate of supply of, or the termination of the supply of, the bypass gaseous exhaust material 60 to the another unit operation is effected when the pressure of the gaseous exhaust material 18 is below a predetermined set point pressure, wherein the decrease in pressure of the gaseous exhaust material 18 to below the predetermined set point pressure is effected in response to an initiation of the supply of, or an increase to the molar rate of supply of, the gaseous exhaust material reaction zone supply 24 to the reaction zone feed material 22, which is effected by the controller in response to the determination that the sensed phototrophic biomass concentration within the reaction zone is below a predetermined low phototrophic biomass concentration value. In such embodiments, upon the controller determining that the sensed phototrophic biomass concentration within the reaction zone by the cell counter 47 is below the predetermined low phototrophic biomass concentration value, the controller effects initiation of the supply of, or effects an increase to the molar rate of supply of, the gaseous exhaust material reaction zone supply 24 to the reaction zone feed material 22, as described above. The initiation of the supply of, or the increase to the molar rate of supply of, the gaseous exhaust material reaction zone supply 24 to the reaction zone feed material 22 effects a corresponding decrease in pressure upstream of the flow control element 50 such that the pressure of the gaseous exhaust material 18 becomes disposed below the predetermined set point pressure. When the pressure of the gaseous exhaust material is below the predetermined minimum pressure, the forces biasing closure of a closure element 64 (such as a valve), disposed between the gaseous exhaust material producing process 20 and the another unit operation, exceed the fluid pressure forces acting to open the closure element 64, and there is effected a decrease in the opening of, or a closure of, the closure element 64. This decrease in the opening of, or the closure of, the closure element 64, effects the decrease to the molar rate of supply of, or the termination of the supply of, the bypass gaseous exhaust material 60 to the another unit operation.

In some embodiments, for example, the phototrophic biomass concentration within the reaction zone is above a predetermined high phototrophic biomass concentration value. In these circumstances, upon the controller comparing a received signal from the cell counter 47, which is representative of the phototrophic biomass concentration within the reaction zone 10, to a target value (i.e., the predetermined high phototrophic biomass concentration value), and determining that the phototrophic biomass concentration within the reaction zone 10 is above the high phototrophic biomass concentration value, the controller responds by effecting reduction of the molar rate of supply of, or effecting termination of the supply of, the gaseous exhaust material reaction zone supply 24 to the reaction zone feed material 22. In some embodiments, for example, this is effected by actuating the flow control element 50 with the controller.

In those embodiments where the outlet of the gaseous exhaust material producing process 20 is co-operatively disposed with another unit operation to effect supply of bypass gaseous exhaust material 60 to the another unit operation, in some of these embodiments, for example, upon the controller comparing a received signal from the cell counter 47, which is representative of the phototrophic biomass concentration within the reaction zone 10, to the predetermined high phototrophic biomass concentration value, and determining that the phototrophic biomass concentration within the reaction zone 10 is above the high phototrophic biomass concentration value, the controller further responds by effecting initiation of the supply of, or effecting an increase to the molar rate of supply of, the bypass gaseous exhaust material 60 to the another unit operation. In some embodiments, for example, the initiation of the supply, or the increase to the molar rate of supply of, the bypass gaseous exhaust material 60 to the another unit operation is effected by the controller by actuation of a valve disposed between the gaseous exhaust material producing process 20 and the another unit operation.

Also in those embodiments where the outlet of the gaseous exhaust material producing process 20 is co-operatively disposed with another unit operation to effect supply of bypass gaseous exhaust material 60 to the another unit operation, in other ones of these embodiments, for example, the initiation of the supply of, or the increase to the molar rate of supply of, the bypass gaseous exhaust material 60 to the another unit operation is effected when the pressure of the gaseous exhaust material 18 is above a predetermined set point pressure, wherein the increase in pressure of the gaseous exhaust material 18 to above the predetermined set point pressure is effected in response to the reduction of the molar rate of supply of, or the termination of the supply of, the gaseous exhaust material reaction zone supply 24 to the reaction zone feed material 22, which is effected by the controller in response to the determination that the sensed phototrophic biomass concentration within the reaction zone is above a predetermined high phototrophic biomass concentration value. In such embodiments, upon the controller determining that the sensed phototrophic biomass concentration within the reaction zone by the cell counter 47 is above the predetermined high phototrophic biomass concentration value, the controller effects a reduction of the molar rate of supply of, or effects termination of the supply of, the gaseous exhaust material reaction zone supply 24 to the reaction zone feed material 22, as described above. The reduction of the molar rate of supply of, or the termination of the supply of, the gaseous exhaust material reaction zone supply 24 to the reaction zone feed material 22 effects a corresponding increase in pressure upstream of the flow control element 50 such that the pressure of the gaseous exhaust material 18 becomes disposed above the predetermined set point pressure. When the pressure of the gaseous exhaust material is above the predetermined set point pressure, the forces biasing closure of a closure element 64 (such as a valve), disposed between the gaseous exhaust material producing process 20 and the another unit operation, are exceeded by the fluid pressure forces acting to open the closure element 64, and there is effected an initiation of the opening of, or an increase to the opening of, the closure element 64. This initiation of the opening of, or the increase to the opening of, the closure element 64, effects the initiation of the supply of, or the increase to the molar rate of supply of, the bypass gaseous exhaust material 60 to the another unit operation.

In some embodiments, for example, the modulating of the bypass gaseous exhaust material 60 to the another unit operation is effected while the modulating of the discharge of the gaseous exhaust material 18 is being effected. In this respect, in some embodiments, for example, the initiation of the supply of, or the increase to the molar rate of supply of, the bypass gaseous exhaust material 60 to the another unit operation is effected while the decrease in the molar rate of supply of, or the termination of the supply of, the gaseous exhaust material reaction zone supply 24 to the reaction zone feed material 22 is being effected. Also in this respect, the decrease to the molar rate of supply of, or the termination of the supply of, the bypass gaseous exhaust material 60 to the another unit operation is effected while the initiation of the supply of, or the increase in the molar rate of supply of, the gaseous exhaust material reaction zone supply 24 to the reaction zone feed material 22 is being effected.

In some embodiments, for example, the flow control element 50 is a flow control valve. In some embodiments, for example, the flow control element 50 is a three-way valve which also regulates the supply of a supplemental gas-comprising material 48, which is further described below. In some embodiments, for example, the closure element 64 is any one of a valve, a damper, or a stack cap.

In some embodiments, for example, when the reaction zone feed material 22 is supplied to the reaction zone 10 as a flow of the reaction zone feed material 22, the flowing of the reaction zone feed material 22 is at least partially effected by a prime mover 38. For such embodiments, examples of a suitable prime mover 38 include a blower, a compressor, a pump (for pressurizing liquids including the gaseous exhaust material reaction zone supply 24), and an air pump. In some embodiments, for example, the prime mover 38 is a variable speed blower and the prime mover 38 also functions as the flow control element 50 which is configured to selectively control the flow rate of the reaction zone feed material 22 and define such flow rate.

In some embodiments, for example, the another unit operation is a smokestack 62. The smokestack 62 is configured to receive the bypass gaseous exhaust material 60 supplied from the outlet of the gaseous exhaust material producing process 20. When operational, the bypass gaseous exhaust material 60 is disposed at a pressure that is sufficiently high so as to effect flow through the smokestack 62. In some of these embodiments, for example, the flow of the bypass gaseous exhaust material 60 through the smokestack 62 is directed to a space remote from the outlet of the gaseous exhaust material producing process 20. Also in some of these embodiments, for example, the bypass gaseous exhaust material 60 is supplied from the outlet when the pressure of the gaseous exhaust material 18 exceeds a predetermined maximum pressure. In such embodiments, for example, the exceeding of the predetermined maximum pressure by the gaseous exhaust material 18 effects an opening of the closure element 64, to thereby effect supply of the bypass gaseous exhaust material 60.

In some embodiments, for example, the smokestack 62 is provided to direct at least a fraction of the flow of a gaseous exhaust material 18 to a space remote from the outlet which discharges the gaseous exhaust material 18 from the gaseous exhaust material producing process 20, in response to a sensed carbon dioxide processing capacity indicator which is suggestive of a capacity of the reaction zone 10 for receiving a decreased molar rate of supply of carbon dioxide from the gaseous exhaust material reaction zone supply 24, so as to mitigate against a gaseous discharge of an unacceptable carbon dioxide concentration to the environment.

In some embodiments, for example, the smokestack 62 is an existing smokestack 62 which has been modified to accommodate lower throughput of gaseous flow as provided by the bypass gaseous exhaust material 60. In this respect, in some embodiments, for example, an inner liner is inserted within the smokestack 62 to accommodate the lower throughput.

In some embodiments, for example, the another unit operation is a separator which effects removal of carbon dioxide from the bypass gaseous exhaust material 60. In some embodiments, for example, the separator is a gas absorber.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and when a carbon dioxide processing capacity indicator is sensed in the reaction zone 10 which is suggestive of a capacity of the reaction zone 10 for receiving a decreased molar rate of supply of carbon dioxide (for example, a sensed pH within the reaction zone that is below a predetermined low pH value, or a sensed phototrophic biomass concentration within the reaction zone that is above a predetermined high phototrophic biomass concentration value), and the modulating of the discharge of the gaseous exhaust material 18, in response to the sensing of the carbon dioxide processing capacity indicator which is suggestive of a capacity of the reaction zone 10 for receiving a decreased molar rate of supply of carbon dioxide, includes reducing the molar rate of supply of, or terminating the supply of, the gaseous exhaust material reaction zone supply 24 to the reaction zone feed material 22, the process further includes initiating the supply, or increasing the molar rate of supply, of a supplemental gas-comprising material 48 to the reaction zone feed material 22. The molar concentration of carbon dioxide, if any, of the supplemental gas-comprising material 48 is lower than the molar concentration of carbon dioxide of the gaseous exhaust material reaction zone supply 24. In some embodiments, for example, the molar concentration of carbon dioxide of the supplemental gas material 48 is less than 3 volume % based on the total volume of the supplemental gas material 48. In some embodiments, for example, the molar concentration of carbon dioxide of the supplemental gas material 48 is less than 1 (one) volume % based on the total volume of the supplemental gas material 48. In some embodiments, for example, the reaction zone feed material 22 is a gaseous material. In some embodiments, for example, the reaction zone feed material 22 includes a dispersion of gaseous material in a liquid material.

In some embodiments, for example, the molar supply rate reduction, or the termination of the supply, of the gaseous exhaust material reaction zone supply 24 to the reaction zone feed material 22 effected by the modulating of the discharge of the gaseous exhaust material 18, co-operates with the supplying of the supplemental gas-comprising material 48 to the reaction zone feed material 22 to effect a reduction in the molar rate of supply of, or the termination of supply of, carbon dioxide to the reaction zone 10 (through the reaction zone feed material 22). In some embodiments, for example, the initiation of the supply of, or the increase to the molar rate of supply of, the bypass gaseous exhaust material 60 to the another unit operation is effected while the decrease in the molar rate of supply of, or the termination of the supply of, the gaseous exhaust material reaction zone supply 24 to the reaction zone feed material 22 is being effected, and while the initiating of the supply of, or the increasing of the molar rate of supply of, the supplemental gas-comprising material 48 to the reaction zone feed material 22 is being effected.

In some of these embodiments, and as described above, the flow control element 50 is a three-way valve, and is operative to modulate supply of the supplemental gas-comprising material 48 in combination with the modulation of the discharge of the gaseous exhaust material 18, in response to the carbon dioxide processing capacity indicator. In this respect, when the carbon dioxide processing capacity indicator is sensed in the reaction zone 10 which is suggestive of a capacity of the reaction zone 10 for receiving a decreased molar rate of supply of carbon dioxide (for example, a sensed pH within the reaction zone that is below a predetermined low pH value, or a sensed phototrophic biomass concentration within the reaction zone that is above a predetermined high phototrophic biomass concentration value), the controller responds by actuating the valve 50 to initiate the supply of, or increase the molar rate of supply of, the supplemental gas-comprising material 48. In some embodiments, while the supplemental gas-comprising material 48 is being supplied to the reaction zone feed material 22, when a carbon dioxide processing capacity indicator is sensed in the reaction zone 10 which is suggestive of a capacity of the reaction zone 10 for receiving an increased molar rate of supply of carbon dioxide (for example, a sensed pH within the reaction zone that is above a predetermined high pH value, or a sensed phototrophic biomass concentration within the reaction zone that is below a predetermined low phototrophic biomass concentration value), the controller responds by actuating the valve 50 to reduce the molar rate of supply of, or terminate the supply of, the supplemental gas-comprising material 48.

In some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, and there is effected a reduction in the molar rate of supply of, or the termination of the supply of, the gaseous exhaust material reaction zone supply 24 to the reaction zone feed material 22, the process further includes initiating the supply, or increasing the molar rate of supply, of a supplemental gas-comprising material 48 to the reaction zone feed material 22.

In some of these embodiments, the reduction in the molar rate of supply of, or the termination of the supply of, the gaseous exhaust material reaction zone supply 24 to the reaction zone feed material 22 is effected in response to the sensing of the carbon dioxide processing capacity indicator which is suggestive of a capacity of the reaction zone 10 for receiving a decreased molar rate of supply of carbon dioxide, as described above. In some embodiments, for example, the corresponding initiating of the supply, or the corresponding increasing of the molar rate of supply, of a supplemental gas-comprising material 48 to the reaction zone feed material 22 is effected also in response to the sensing of the carbon dioxide processing capacity indicator which is suggestive of a capacity of the reaction zone 10 for receiving a decreased molar rate of supply of carbon dioxide. In some embodiments, for example, the corresponding initiating of the supply, or the corresponding increasing of the molar rate of supply, of a supplemental gas-comprising material 48 to the reaction zone feed material 22 is effected in response to the sensing of the reduction in the molar rate of supply of, or the termination of the supply of, the gaseous exhaust material reaction zone supply 24 to the reaction zone feed material 22 (effected in response to the sensing of the carbon dioxide processing capacity indicator which is suggestive of a capacity of the reaction zone 10 for receiving a decreased molar rate of supply of carbon dioxide).

In other ones of these embodiments, the reduction in the molar rate of supply of, or the termination of the supply of, the gaseous exhaust material reaction zone supply 24 to the reaction zone feed material 22 is effected by a reduction in the molar rate of supply of the gaseous exhaust material 18 to the gaseous exhaust material reaction zone supply 24, such as that effected by a reduced rate of production of the gaseous exhaust material 18 by the gaseous exhaust material producing process 20. In some embodiments, for example, the exposing of the phototrophic biomass disposed in the reaction zone 10 to photosynthetically active light radiation is effected while the initiation of the supply of, or the increasing of the molar rate of supply of, the supplemental gas-comprising material 48 to the reaction zone feed material 22 is being effected. In some embodiments, for example, the modulation of the supply of the supplemental gas-comprising material 48 to the reaction zone feed material 22 is effected by the flow control element 50, for example, upon actuation by the controller. In some embodiments, the actuation by the controller is effected when a sensed flow rate of the gaseous exhaust material reaction zone supply 24 by a flow sensor, which is representative of a current molar flow rate of the gaseous exhaust material 24, or a sensed flow rate of the gaseous exhaust material 18 by a flow sensor, which is representative of a current molar flow rate of the gaseous exhaust material 18, is compared to a previously sensed molar flow rate of the corresponding material flow, and it is determined that there has been a decrease in the molar flow rate of the corresponding material.

With respect to any of the above-described embodiments of the process where there is the reduction in the molar rate of supply of, or the termination of supply of, the gaseous exhaust material reaction zone supply 24 to the reaction zone feed material 22 is effected, and where there is initiated the supply, or the increase to the molar rate of supply, of the supplemental gas-comprising material 48 to the reaction zone feed material 22, in some of these embodiments, for example, the initiation of the supply of, or the increasing of the molar rate of supply of, the supplemental gas-comprising material 48 to the reaction zone feed material 22 at least partially compensates for the reduction in molar supply rate of material, or the termination of any material supply, to the reaction zone feed material 22 which is effected by the reduction in the molar rate of supply of, or by the termination of supply of, the gaseous exhaust material reaction zone supply 24 to the reaction zone feed material 22. In some embodiments, for example, the compensation for the reduction in molar supply rate of material, or for the termination of any material supply, to the reaction zone feed material 22 which is effected by the reduction in the molar rate of supply of, or by the termination of supply of, the gaseous exhaust material reaction zone supply 24 to the reaction zone feed material 22., as effected by the initiation of the supply of, or the increasing of the molar rate of supply of, the supplemental gas-comprising material 48, effects substantially no change to the molar rate of flow of reaction zone feed material 22 to the reaction zone 10.

The combination of: (a) the reduction of the molar rate of supply of, or the termination of supply of, the gaseous exhaust material reaction zone supply 24 to the reaction zone feed material 22, and (b) the initiation of the supply of, or the increase to the molar rate of supply of, the supplemental gas-comprising material 48 to the reaction zone feed material 22, mitigates against the reduced agitation of the reaction zone 10 attributable to the reduction in the molar rate of supply of, or the termination of supply of, the gaseous exhaust material reaction zone supply 24 to the reaction zone feed material 22. In some embodiments, for example, the reaction zone feed material 22 is flowed to the reaction zone 10 and effects agitation of material in the reaction zone such that any difference in phototrophic biomass concentration between two points in the reaction zone 10 is less than 20%. In some embodiments, for example, the effected agitation is such that any difference in phototrophic biomass concentration between two points in the reaction zone 10 is less than 10%. The supply of the supplemental gas-comprising material 48 is provided to mitigate against the creation of a phototrophic biomass concentration gradient between any two points in the reaction zone above a desired maximum.

In some embodiments, for example, the supplemental gas-comprising material 48 is a gaseous material. In some of these embodiments, for example, the supplemental gas-comprising material 48 includes a dispersion of gaseous material in a liquid material. In some of these embodiments, for example, the supplemental gas-comprising material 48 includes air. In some of these embodiments, for example, the supplemental gas-comprising material 48 is provided as a flow.

In some circumstances, it is desirable to grow phototrophic biomass using carbon dioxide of the gaseous exhaust material 18 being discharged from the gaseous exhaust material producing process 20, but the molar concentration of carbon dioxide in the discharged gaseous exhaust material 18 is excessive for effecting a desired growth rate of the phototrophic biomass. In this respect, the phototrophic biomass responds adversely when exposed to the reaction zone feed material 22 which is supplied by the gaseous exhaust material reaction zone supply 24 of the gaseous exhaust material 18, by virtue of the carbon dioxide concentration of the reaction zone feed material 22, which is attributable to the molar concentration of carbon dioxide of the gaseous exhaust reaction zone supply 24.

In other circumstances, it is necessary to supply the reaction zone feed material 22 with the supplemental carbon dioxide supply 92, as described above. In some of these embodiments, the supplemental carbon dioxide supply 92 includes a relatively high concentration of carbon dioxide (such as greater than 90 mol % carbon dioxide based on the total moles of supplemental carbon dioxide supply 92). The phototrophic biomass responds adversely when exposed to the reaction zone feed material 22 which is supplied by the supplemental carbon dioxide supply 92, by virtue of the carbon dioxide concentration of the reaction zone feed material 22, which is attributable to the molar concentration of carbon dioxide of the supplemental carbon dioxide supply 92.

In this respect, in some embodiments, for example, while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10, the process further includes, supplying the reaction zone feed material 22 with a supplemental gaseous dilution agent 90, wherein the molar concentration of carbon dioxide of the supplemental gaseous dilution agent 90 is less than the molar concentration of carbon dioxide of the gaseous exhaust material reaction zone supply 24 which is supplying the reaction zone feed material 22. The supplying of the supplemental gaseous dilution agent 90 to the reaction zone feed material 22 provides a molar concentration of carbon dioxide in the reaction zone feed material 22 being supplied to the reaction zone 10 that is below a predetermined maximum carbon dioxide molar concentration value. In some embodiments, for example, the predetermined maximum carbon dioxide concentration value is 30 mol % based on the total moles of the reaction zone feed material 22. In some embodiments, for example, the predetermined maximum carbon dioxide concentration value is 20 mol % based on the total moles of the reaction zone feed material 22. In some of these embodiments, for example, the supplying of the supplemental gaseous dilution agent 90 to the reaction zone feed material 22 effects dilution of the reaction zone feed material 22 with respect to molar concentration of carbon dioxide (i.e., effects reduction of molar concentration of carbon dioxide in the reaction zone feed material 22). In some of these embodiments, the exposing of the phototrophic biomass disposed in the reaction zone 10 to photosynthetically active light radiation is effected while the supplying of the reaction zone feed material 22 with a supplemental gaseous dilution agent 90 is being effected.

In those embodiments where the supplemental gaseous dilution agent 90 is supplied to the reaction zone feed material 22 while the supplemental carbon dioxide supply 92 is also supplying the reaction zone feed material 22, the supplying of the supplemental gaseous dilution agent 90 to the reaction zone feed material 22 provides a molar concentration of carbon dioxide in the reaction zone feed material 22 being supplied to the reaction zone 10 that is at least 80% of the molar concentration of carbon dioxide of the gaseous exhaust material reaction zone supply 24 supplying the reaction zone feed material 22 before the supply of the supplemental gaseous dilution agent 90 had been initiated in response to the sensing of an indication of a decrease in the molar rate of supply of carbon dioxide being supplied to the reaction zone feed material 22 by the gaseous exhaust material producing process 20 as gaseous exhaust material reaction zone supply 24. In some embodiments, for example, the supplying of the supplemental gaseous dilution agent 90 to the reaction zone feed material 22 provides a molar concentration of carbon dioxide in the reaction zone feed material 22 being supplied to the reaction zone 10 that is at least 90% of the molar concentration of carbon dioxide of the gaseous exhaust material reaction zone supply 24 supplying the reaction zone feed material 22 before the supply of the supplemental gaseous dilution agent 90 had been initiated response to the sensing of an indication of a decrease in the molar rate of supply of carbon dioxide being supplied to the reaction zone feed material 22 by the gaseous exhaust material producing process 20 as gaseous exhaust material reaction zone supply 24. In some embodiments, for example, the supplying of the supplemental gaseous dilution agent 90 to the reaction zone feed material 22 provides a molar concentration of carbon dioxide in the reaction zone feed material 22 being supplied to the reaction zone 10 that is at least 95% of the molar concentration of carbon dioxide of the gaseous exhaust material reaction zone supply 24 supplying the reaction zone feed material 22 before the supply of the supplemental gaseous dilution agent 90 had been initiated response to the sensing of an indication of a decrease in the molar rate of supply of carbon dioxide being supplied to the reaction zone feed material 22 by the gaseous exhaust material producing process 20 as gaseous exhaust material reaction zone supply 24.

In some of these embodiments, for example, the reaction zone feed material 22 includes an upstream reaction zone feed material 22A and a downstream reaction zone feed material 22B, wherein the downstream reaction zone feed material 22B is downstream of the upstream reaction zone feed material 22A relative to the reaction zone 10. The supplemental gaseous dilution agent 90 is admixed with the upstream reaction zone feed material 22A to provide the downstream reaction zone feed material 22B such that the molar concentration of carbon dioxide in the downstream reaction zone feed material 22B is less than the molar concentration of carbon dioxide in the upstream reaction zone feed material 22A. In some embodiments, for example, the upstream reaction zone feed material 22A is a gaseous material, and the downstream reaction zone feed material 22B is a gaseous material, and the downstream reaction zone feed material 22B is supplied to the reaction zone 10.

In some embodiments, for example, the supplying of the supplemental gaseous dilution agent 90 to the reaction zone feed material 22 is effected in response to sensing of a molar concentration of carbon dioxide in the gaseous exhaust material 18 being discharged from the carbon dioxide producing process 20 that is greater than a predetermined maximum carbon dioxide molar concentration value. In some embodiments, for example, the predetermined maximum carbon dioxide molar concentration value is 10 volume % based on the total volume of the gaseous exhaust material 18. Because any of the discharged gaseous effluent material 18 that is supplied to the reaction zone feed material 22 is supplied as the gaseous exhaust material reaction zone supply 24, the sensing of the molar concentration of carbon dioxide of the discharged gaseous effluent material 18 includes sensing of the molar concentration of carbon dioxide of the gaseous exhaust material reaction zone supply 24. In this respect, in some embodiments, a carbon dioxide sensor 781 is provided for sensing the molar concentration of carbon dioxide of the gaseous exhaust material 18 being produced, and transmitting a signal representative of the molar concentration of carbon dioxide of the gaseous exhaust material 18 being produced to the controller. Upon the controller comparing a received signal from the carbon dioxide sensor 781 which is representative of a current molar concentration of carbon dioxide of the gaseous exhaust material 18 to a predetermined maximum carbon dioxide molar concentration value, and determining that the molar concentration of carbon dioxide of the gaseous exhaust material 18 is greater than a predetermined maximum carbon dioxide molar concentration value, the controller actuates opening of a control valve 901 which effects supply of the supplemental gaseous dilution agent 90 to the reaction zone feed material 22.

In some embodiments, the reaction zone feed material 22 is supplied to the reaction zone 10 as a flow. In some embodiments, for example, the supplemental gaseous dilution agent 90 is gaseous material. In some embodiments, for example, the supplemental gaseous dilution agent 90 includes air. In some embodiments, for example, the supplemental gaseous dilution agent 90 is being supplied to the reaction zone feed material 22 as a flow. In some embodiments, for example, the supplemental gaseous dilution agent 90 is a gaseous material and is supplied as a flow for admixing with the upstream reaction zone feed material 22A.

The reaction mixture disposed in the reaction zone 10 is exposed to photosynthetically active light radiation so as to effect photosynthesis. The photosynthesis effects growth of the phototrophic biomass. In some embodiments, for example, there is provided the carbon dioxide-enriched phototrophic biomass disposed in the aqueous medium, and the carbon dioxide-enriched phototrophic biomass disposed in the aqueous medium is exposed to photosynthetically active light radiation so as to effect photosynthesis.

In some embodiments, for example, the light radiation is characterized by a wavelength of between 400-700 nm. In some embodiments, for example, the light radiation is in the form of natural sunlight. In some embodiments, for example, the light radiation is provided by an artificial light source 14. In some embodiments, for example, light radiation includes natural sunlight and artificial light.

In some embodiments, for example, the intensity of the provided light is controlled so as to align with the desired growth rate of the phototrophic biomass in the reaction zone 10. In some embodiments, regulation of the intensity of the provided light is based on measurements of the growth rate of the phototrophic biomass in the reaction zone 10. In some embodiments, regulation of the intensity of the provided light is based on the molar rate of supply of carbon dioxide to the reaction zone feed material 22.

In some embodiments, for example, the light is provided at pre-determined wavelengths, depending on the conditions of the reaction zone 10. Having said that, generally, the light is provided in a blue light source to red light source ratio of 1:4. This ratio varies depending on the phototrophic organism being used. As well, this ratio may vary when attempting to simulate daily cycles. For example, to simulate dawn or dusk, more red light is provided, and to simulate mid-day condition, more blue light is provided. Further, this ratio may be varied to simulate artificial recovery cycles by providing more blue light.

It has been found that blue light stimulates algae cells to rebuild internal structures that may become damaged after a period of significant growth, while red light promotes algae growth. Also, it has been found that omitting green light from the spectrum allows algae to continue growing in the reaction zone 10 even beyond what has previously been identified as its “saturation point” in water, so long as sufficient carbon dioxide and, in some embodiments, other nutrients, are supplied.

With respect to artificial light sources, for example, a suitable artificial light source 14 includes submersible fiber optics, light-emitting diodes, LED strips, and fluorescent lights. Any LED strips known in the art can be adapted for use in the process. In the case of the submersible LEDs, the design includes the use of solar powered batteries to supply the electricity. In the case of the submersible LEDs, in some embodiments, for example, energy sources include alternative energy sources, such as wind, photovoltaic cells, fuel cells, etc. to supply electricity to the LEDs.

With respect to those embodiments where the reaction zone 10 is disposed in a photobioreactor 12 which includes a tank, in some of these embodiments, for example, the light energy is provided from a combination of sources, as follows. Natural light source 16 in the form of solar light is captured though solar collectors and filtered with custom mirrors that effect the provision of light of desired wavelengths to the reaction zone 10. The filtered light from the solar collectors is then transmitted through light guides or fiber optic materials into the photobioreactor 12, where it becomes dispersed within the reaction zone 10. In some embodiments, in addition to solar light, the light tubes in the photobioreactor 12 contains high power LED arrays that can provide light at specific wavelengths to either complement solar light, as necessary, or to provide all of the necessary light to the reaction zone 10 during periods of darkness (for example, at night). In some embodiments, with respect to the light guides, for example, a transparent heat transfer medium (such as a glycol solution) is circulated through light guides within the photobioreactor 12 so as to regulate the temperature in the light guides and, in some circumstances, provide for the controlled dissipation of heat from the light guides and into the reaction zone 10. In some embodiments, for example, the LED power requirements can be predicted and, therefore, controlled, based on trends observed with respect to the gaseous exhaust material 18, as these observed trends assist in predicting future growth rate of the phototrophic biomass.

In some embodiments, the exposing of the reaction mixture to photosynthetically active light radiation is effected while the supplying of the reaction feed material 22 is being effected.

In some embodiments, for example, the growth rate of the phototrophic biomass is dictated by the available gaseous exhaust material reaction zone supply 24. In turn, this defines the nutrient, water, and light intensity requirements to maximize phototrophic biomass growth rate. In some embodiments, for example, a controller, e.g. a computer-implemented system, is provided to be used to monitor and control the operation of the various components of the process disclosed herein, including lights, valves, sensors, blowers, fans, dampers, pumps, etc.

Reaction zone product 500 is discharged from the reaction zone. The reaction zone product 500 includes phototrophic biomass-comprising product 58. In some embodiments, for example, the phototrophic biomass-comprising product 58 includes at least a fraction of the contents of the reaction zone 10. In this respect, the discharge of the reaction zone product 500 effects harvesting of the phototrophic biomass. In some embodiments, for example, the reaction zone product 500 also includes a reaction zone gaseous effluent product.

In one aspect, there is provided a process for growing a phototrophic biomass in a reaction zone 10 that includes modulating of the molar rate of discharge of the reaction zone product 500 based on the sensing of a process parameter.

The reaction mixture, in the form of a production purpose reaction mixture that is operative for effecting photosynthesis upon exposure to photosynthetically active light radiation, is provided. The reaction zone includes the production purpose reaction mixture. The production purpose reaction mixture includes phototrophic biomass in the form of production purpose phototrophic biomass that is operative for growth within the reaction zone 10. In this respect, a reaction zone concentration of production purpose phototrophic biomass is provided in the reaction zone 10. The growth of the production purpose phototrophic biomass includes that which is effected by the photosynthesis. While growth of the production purpose phototrophic biomass is effected in the reaction zone 10, and while reaction zone product is discharging from the reaction zone, and when a sensed value of a process parameter is different than a target value of the process parameter, the process includes modulating the molar rate of discharge of the reaction zone product from the reaction zone, wherein the target value of the process parameter is based upon a desired molar growth rate of the production purpose phototrophic biomass within the reaction zone 10. The reaction zone product 500 includes a portion of the production purpose phototrophic biomass.

In some embodiments, for example, the target value of the process parameter corresponds to the desired molar growth rate of the production purpose phototrophic biomass within the reaction zone.

In some embodiments, for example, the effected growth of the production purpose phototrophic biomass in the reaction zone is being effected within 10% of the desired growth rate of the production purpose phototrophic biomass within the reaction zone 10. In some embodiments, the effected growth of the production purpose phototrophic biomass in the reaction zone is being effected within 5% of the desired growth rate of the production purpose phototrophic biomass within the reaction zone 10. In some embodiments, the effected growth of the production purpose phototrophic biomass in the reaction zone is being effected within 1% of the desired growth rate of the production purpose phototrophic biomass within the reaction zone 10.

In some embodiments, for example, the modulating is effected in response to comparing of the sensed value of the process parameter to the target value of the process parameter.

In some embodiments, for example, the process further includes sensing a process parameter to provide the sensed value of the process parameter.

In some embodiments, for example, the sensed value of the process parameter is representative of the reaction zone concentration of the production purpose phototrophic biomass. In this respect, in some of these embodiments, for example, the sensed value of the process parameter is the reaction zone concentration of the production purpose phototrophic biomass. In other ones of these embodiments, for example, the sensed value of the process parameter is the concentration of the production purpose phototrophic biomass in the reaction zone product 500 (such as the phototrophic biomass-comprising product 58). In some embodiments, for example, the sensing of the concentration is effected by a cell counter 47. For example, a suitable cell counter is an AS-16F Single Channel Absorption Probe supplied by optek-Danulat, Inc. of Germantown, Wis., U.S.A. Other suitable devices for sensing a phototrophic biomass concentration indication include other light scattering sensors, such as a spectrophotometer. As well, the phototrophic biomass concentration can be sensed manually, and then input manually into a controller for effecting the desired response.

In some embodiments, for example, the effecting of the growth of the phototrophic biomass includes supplying carbon dioxide to the reaction zone 10 and exposing the production purpose reaction mixture to photosynthetically active light radiation. In some embodiments, for example, the supplied carbon dioxide is supplied from the gaseous exhaust material 18 of the gaseous exhaust material producing process 20. In some embodiments, for example, the supplied carbon dioxide is supplied from the gaseous exhaust material 18 of the gaseous exhaust material producing process 20 while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10. In this respect, in some embodiments, for example, the carbon dioxide is supplied to the reaction zone 10 while the growth is being effected, wherein at least a fraction of the carbon dioxide being supplied to the reaction zone is supplied from a gaseous exhaust material while the gaseous exhaust material is being discharged from a gaseous exhaust material producing process.

In some embodiments, for example, the production purpose reaction mixture further includes water and carbon dioxide.

In some embodiments, for example, the desired molar growth rate of the production purpose phototrophic biomass within the reaction zone 10 is at least 90% of the maximum molar growth rate of the production purpose phototrophic biomass within the reaction zone 10. In some embodiments, for example, the desired molar growth rate is at least 95% of the maximum molar growth rate of the production purpose phototrophic biomass within the reaction zone 10. In some embodiments, for example, the desired molar growth rate is at least 99% of the maximum molar growth rate of the production purpose phototrophic biomass within the reaction zone 10. In some embodiments, for example, the desired molar growth rate is the maximum molar growth rate of the production purpose phototrophic biomass within the reaction zone 10.

In some embodiments, for example, the reaction zone is disposed within a photobioreactor, and the reaction zone product includes an overflow of the phototrophic biomass-comprising product 58 that is discharged from the photobioreactor. In some embodiments, for example, the overflow is effected in response to the supplying of an aqueous feed material 4 to the reaction zone 10.

In some embodiments, for example, the aqueous feed material 4 includes substantially no phototrophic biomass. In other embodiments, for example, the aqueous feed material includes phototrophic biomass at a concentration less than the reaction zone concentration of phototrophic biomass.

In some embodiments, for example, with respect to the aqueous feed material 4, the aqueous feed material 4 is supplied as a flow from a source 6 of aqueous feed material 4. For example, the flow is effected by a prime mover, such as pump. In some embodiments, for example, the aqueous feed material includes the supplemental aqueous material supply 44. As described above, in some embodiments, for example, at least a fraction of the supplemental aqueous material supply 44 is supplied from a container 28. In this respect, in those embodiments where the supplemental aqueous material supply 44 is included within the aqueous feed material, the container functions as the source 6 of the aqueous feed material 4.

In some embodiments, for example, the aqueous feed material 4 includes the supplemental nutrient supply 42 and the supplemental aqueous material supply 44. In some of these embodiments, the aqueous feed material 4 is supplied to the reaction zone feed material 22 before the reaction zone feed material 22 is introduced to the reaction zone 10. In this respect, and referring to FIG. 2, and as described above, in some of these embodiments, the supplemental nutrient supply 42 and the supplemental aqueous material supply 44 are supplied to the reaction zone feed material 22 through the sparger 40 before being supplied to the reaction zone 10.

In some embodiments, for example, when the sensed value of the process parameter is representative of a molar concentration of phototrophic biomass in the reaction zone 10, and the sensed molar concentration of phototrophic biomass in the reaction zone 10 is less than the target value, the modulating includes effecting a decrease in the molar rate of discharge of the reaction zone product 500 from the reaction zone 10. In some of these embodiments, for example, the reaction zone product 500 that is discharged from the reaction zone includes an overflow 59 from a photobioreactor 12, and the decrease in the molar rate of discharge of the reaction zone product 500 from the reaction zone 10 is effected by effecting a decrease in the molar rate of supply of, or termination of the supply of, the aqueous feed material 4 to the reaction zone 10. In this respect, when the phototrophic biomass-comprising product 58 is discharged as an overflow, in some embodiments, for example, when the sensed value of the process parameter is representative of a molar concentration of phototrophic biomass in the reaction zone 10, upon comparing the molar concentration of phototrophic biomass in the reaction zone 10, which is sensed by the cell counter 47, with the target value, and determining that the sensed molar concentration is less than the target value, the controller responds by effecting a decrease in the molar rate of supply of, or termination of supply of, the aqueous feed material 4 to the reaction zone 10, which thereby effects a decrease in the molar rate of discharge of, or termination of, the phototrophic biomass-comprising product 58 from the reaction zone 10. In some embodiments, for example, the decrease in the molar rate of supply of the aqueous feed material 4 to the reaction zone 10 is effected by the controller by actuating a decrease in the opening of a control valve 441 that is disposed in a fluid passage that facilitates supply of a flow of the aqueous feed material 4 from the source 6 to the reaction zone 10. In some embodiments, for example, the termination of supply of the aqueous feed material 4 to the reaction zone 10 is effected by the controller by actuating closure of a control valve 441 that is disposed in a fluid passage that facilitates supply of a flow of the aqueous feed material 4 from the source 6 to the reaction zone 10. In some embodiments, for example, the flow of the aqueous feed material 4 is being effected by a prime mover, such as a pump 281. In some embodiments, for example, the flow of the aqueous feed material 4 is being effected by gravity. In some embodiments, for example, the aqueous feed material 4 includes the supplemental aqueous material supply 44 which is supplied from the container 28. In some embodiments, the aqueous feed material 4 is the supplemental aqueous material supply 44 which is supplied from the container 28. In some of these embodiments, for example, the supplemental aqueous material supply 44 is supplied from the container 28 by the pump 281, and in other ones of these embodiments, for example, the supplemental aqueous material supply 44 is supplied from the container 28 by gravity. In some embodiments, for example, where a prime mover (such as the pump 281) is provided for effecting the flow of the aqueous feed material 4 to the reaction zone 10, the decrease in the molar rate of supply of the aqueous feed material 4 to the reaction zone 10 is effected by the controller actuating a decrease to the kinetic energy being imparted by the prime mover 281 to the aqueous feed material 4. In some embodiments, for example, where a prime mover (such as the pump 281) is provided for effecting the flow of the aqueous feed material 4 to the reaction zone 10, the termination of supply of the aqueous feed material 4 to the reaction zone 10 is effected by the controller actuating stoppage of the prime mover.

In some embodiments, for example, when the sensed value of the process parameter is representative of a molar concentration of phototrophic biomass in the reaction zone 10, and the sensed molar concentration of phototrophic biomass in the reaction zone 10 is greater than the target value, the modulating includes effecting an increase in the molar rate of discharge of the reaction zone product 500 from the reaction zone 10. In some of these embodiments, for example, the reaction zone product 500 that is discharged from the reaction zone 10 includes an overflow 59 of the phototrophic biomass-comprising product 58 from a photobioreactor, and the increase in the molar rate of discharge of the reaction zone product 500 from the reaction zone 10 is effected by effecting initiation of supply of, or an increase in the molar rate of supply of, the aqueous feed material 4 to the reaction zone 10. In this respect, when the phototrophic biomass-comprising product 58 is discharged as an overflow 59, in some embodiments, for example, when the sensed value of the process parameter is representative of a molar concentration of phototrophic biomass in the reaction zone 10, upon comparing a molar concentration of phototrophic biomass in the reaction zone 10, which is sensed by the cell counter 47, with the target value, and determining that the sensed molar concentration is greater than the target value, the controller responds by effecting initiation of supply of, or an increase in the molar rate of supply of, the aqueous feed material 4 to the reaction zone 10, which thereby effects an increase in the molar rate of discharge of the phototrophic biomass-comprising product 58 from the reaction zone 10. In some embodiments, for example, the initiation of supply of the aqueous feed material 4 to the reaction zone 10 is effected by the controller by actuating opening of a control valve 441 that is disposed in a fluid passage that facilitates supply of a flow of the aqueous feed material 4 from the source 6 to the reaction zone 10. In some embodiments, for example, the increase in the molar rate of supply of the aqueous feed material 4 to the reaction zone 10 is effected by the controller by actuating an increase in the opening of a control valve 441 that is disposed in a fluid passage that facilitates supply of a flow of the aqueous feed material 4 from the source 6 to the reaction zone 10. In some embodiments, for example, the flow of the aqueous feed material 4 is being effected by a prime mover, such as a pump 281. In some embodiments, for example, the flow of the aqueous feed material 4 is being effected by gravity. In some embodiments, for example, the aqueous feed material includes the supplemental aqueous material supply 44 which is supplied from the container 28. In some embodiments, for example, the aqueous feed material is the supplemental aqueous material supply 44 which is supplied from the container 28. In some of these embodiments, for example, the supplemental aqueous material supply 44 is supplied from the container 28 by the pump 281, and in other ones of these embodiments, for example, the supplemental aqueous material supply 44 is supplied from the container 28 by gravity. In some embodiments, for example, where a prime mover (such as the pump 281) is provided for effecting the flow of the aqueous feed material 4 to the reaction zone 10, the initiation of supply of the aqueous feed material 4 to the reaction zone 10 is effected by the controller actuating operation of the prime mover. In some embodiments, for example, where a prime mover (such as the pump 281) is provided for effecting the flow of the aqueous feed material 4 to the reaction zone 10, the increase in the molar rate of supply of the aqueous feed material 4 to the reaction zone 10 is effected by the controller actuating an increase to the kinetic energy being imparted by the prime mover to the aqueous feed material 4.

In some embodiments, for example, the target value is predetermined. In some embodiments, for example, the desired growth rate is predetermined. In this respect, in some of these embodiments, the process further includes effecting the predetermination of the target value. In this respect, an evaluation purpose reaction mixture that is representative of the production purpose reaction mixture and is operative for effecting photosynthesis upon exposure to photosynthetically active light radiation is provided, such that the phototrophic biomass of the evaluation purpose reaction mixture is an evaluation purpose phototrophic biomass that is representative of the production purpose phototrophic biomass. In some embodiments, for example, the production purpose reaction mixture further includes water and carbon dioxide, and the evaluation purpose reaction mixture further includes water and carbon dioxide. While effecting growth of the evaluation purpose phototrophic biomass in the reaction zone 10, and while discharging evaluation purpose product from the reaction zone 10, wherein the evaluation purpose product includes a portion of the evaluation purpose phototrophic biomass, the process further includes:
(i) at least periodically measuring the process parameter to provide a plurality of measured values of the process parameter that have been measured during a time period (“at least periodically” means that the measuring could be done intermittently, at equally spaced intervals or at unequally spaced time intervals, or could be done continuously);
(ii) calculating molar growth rates of the evaluation purpose phototrophic biomass based on the plurality of measured values of the process parameter such that a plurality of molar growth rates of the evaluation purpose phototrophic biomass are determined during the time period; and
(iii) establishing a relationship between the molar growth rate of the evaluation purpose phototrophic biomass and the process parameter, based on the calculated molar growth rates and the measured values of the process parameter upon which the calculated molar growth rates have been based;

such that the established relationship between the molar growth rate of the evaluation purpose phototrophic biomass and the process parameter is representative of a relationship between the molar growth rate of the production purpose phototrophic biomass within the reaction zone 10 and the process parameter, and such that the relationship between the molar growth rate of the production purpose phototrophic biomass within the reaction zone 10 and the process parameter. Based on the relationship between the molar growth rate of the production purpose phototrophic biomass within the reaction zone 10 and the process parameter, the desired molar growth rate is selected, and the target value is determined as being the process parameter at which the desired molar growth rate is being effected, such that the correspondence between the target value and the desired molar growth rate is also thereby effected. In some embodiments, for example, the effected growth of the evaluation purpose phototrophic biomass in the reaction zone 10 is effected while the evaluation purpose reaction mixture is exposed to at least one evaluation purpose growth condition that is representative of a production purpose growth condition to which the production purpose reaction mixture is exposed to while growth of the production purpose phototrophic biomass in the reaction zone 10 is being effected. In some embodiments, for example, the production purpose growth condition is any one of a plurality of production purpose growth conditions including composition of the reaction zone, reaction zone temperature, reaction zone pH, reaction zone light intensity, reaction zone lighting regimes (e.g., variable intensities), reaction zone lighting cycles (e.g., duration of ON/OFF lighting cycles), and reaction zone temperature. In some embodiments, for example, providing one or more evaluation purpose growth conditions, each of which is representative of a production purpose growth condition to which the production purpose reaction mixture is exposed to while growth of the production purpose phototrophic biomass in the reaction zone 10 is being effected, promotes optimization of phototrophic biomass production. In some embodiments, for example, the discharging of the evaluation purpose product from the reaction zone 10 is effected as an overflow, and the overflow is effected in response to supplying of aqueous feed material to the reaction zone 10.

In another aspect, while the phototrophic biomass is growing at or relatively close to the maximum molar growth rate within the reaction zone 10, a molar rate of discharge of the reaction zone product is provided that at least approximates the growth rate of the phototrophic biomass within the reaction zone.

The reaction mixture, in the form of a production purpose reaction mixture that is operative for effecting photosynthesis upon exposure to photosynthetically active light radiation, is provided. The reaction zone includes the production purpose reaction mixture. The production purpose reaction mixture includes phototrophic biomass in the form of production purpose phototrophic biomass that is operative for growth within the reaction zone 10. The growth of the production purpose phototrophic biomass includes that which is effected by the photosynthesis. While growth of the production purpose phototrophic biomass within the reaction zone is effected at a desired molar growth rate, a reaction zone product 500 including production purpose phototrophic biomass is discharged from the reaction zone 10 to provide a molar rate of discharge of the production purpose phototrophic biomass that is within 10% of the desired molar growth rate. The desired molar growth rate is at least 90% of the maximum growth rate of the production purpose phototrophic biomass within the reaction zone 10. In some embodiments, for example, the molar rate of discharge of the production purpose phototrophic biomass that is provided is within 5% of the desired molar growth rate. In some embodiments, for example, the molar rate of discharge of the production purpose phototrophic biomass that is provided is within 1% of the desired molar growth rate. In some embodiments, for example, the desired molar growth rate is at least 95% of the maximum growth rate of the production purpose phototrophic biomass within the reaction zone 10, and in some of these embodiments, for example, the molar rate of discharge of the production purpose phototrophic biomass that is provided is within 5%, such as within 1%, of the desired molar growth rate. In some embodiments, for example, the desired molar growth rate is at least 99% of the maximum growth rate of the production purpose phototrophic biomass within the reaction zone 10, and in some of these embodiments, for example, the molar rate of the phototrophic biomass-comprising product 58 of the production purpose phototrophic biomass that is provided is within 5%, such as within 1%, of the desired molar growth rate.

In some embodiments, for example, the effecting of the growth of the production purpose phototrophic biomass includes supplying carbon dioxide to the reaction zone 10 and exposing the production purpose reaction mixture to photosynthetically active light radiation. In some embodiments, for example, the supplied carbon dioxide is supplied from the gaseous exhaust material 18 of the gaseous exhaust material producing process 20. In some embodiments, for example, the supplied carbon dioxide is supplied from the gaseous exhaust material 18 of the gaseous exhaust material producing process 20 while the gaseous exhaust material 18 is being produced by the gaseous exhaust material producing process 20, and while at least a fraction of the gaseous exhaust material 18 is being supplied to the reaction zone feed material 22 (as the gaseous exhaust material reaction zone supply 24), and while the reaction zone feed material 22 is being supplied to the reaction zone 10. In this respect, in some embodiments, for example, the carbon dioxide is supplied to the reaction zone 10 while the growth is being effected, wherein at least a fraction of the carbon dioxide being supplied to the reaction zone is supplied from a gaseous exhaust material while the gaseous exhaust material is being discharged from a gaseous exhaust material producing process.

In some embodiments, for example, the reaction zone 10 is disposed within a photobioreactor 12, and the reaction zone product 500 is discharged from the reaction zone 10 and includes an overflow from the photobioreactor 12. In some embodiments, for example, the overflow is effected in response to the supplying of an aqueous feed material 4 to the reaction zone 10.

In some embodiments, for example, the reaction zone product 500 is discharged as an overflow of the phototrophic biomass-comprising product 58 while aqueous feed material 4 is being supplied to the reaction zone and reaction zone product 500 is being discharged from the reaction zone 10. In some of these embodiments, for example, the aqueous feed material 4 includes substantially no production purpose phototrophic biomass. In other ones of these embodiments, for example, the aqueous feed material 4 includes production purpose phototrophic biomass at a concentration less than the reaction zone concentration of the production purpose phototrophic biomass.

In some embodiments, for example, with respect to the aqueous feed material 4, the aqueous feed material 4 is supplied as a flow from a source 6 of aqueous feed material 4. For example, the flow is effected by a prime mover, such as pump. In some embodiments, for example, the aqueous feed material includes the supplemental aqueous material supply 44. As described above, in some embodiments, for example, at least a fraction of the supplemental aqueous material supply 44 is supplied from a container 28. In this respect, in those embodiments where the supplemental aqueous material supply 44 is included within the aqueous feed material, the container functions as the source 6 of the aqueous feed material 4.

In some embodiments, for example, the aqueous feed material 4 includes the supplemental nutrient supply 42 and the supplemental aqueous material supply 44. In some of these embodiments, the aqueous feed material 4 is supplied to the reaction zone feed material 22 before the reaction zone feed material 22 is introduced to the reaction zone 10. In this respect, and referring to FIG. 2, and as described above, in some of these embodiments, the supplemental nutrient supply 42 and the supplemental aqueous material supply 44 are supplied to the reaction zone feed material 22 through the sparger 40 before being supplied to the reaction zone 10.

In some embodiments, for example, the maximum molar growth rate of the production purpose phototrophic biomass is predetermined, and the predetermination of the maximum molar growth rate of the production purpose phototrophic biomass includes providing an evaluation purpose reaction mixture that is representative of the production purpose reaction mixture and is operative for effecting photosynthesis upon exposure to photosynthetically active light radiation, such that the phototrophic biomass of the evaluation purpose reaction mixture is an evaluation purpose phototrophic biomass that is representative of the production purpose phototrophic biomass, and while effecting growth of the evaluation purpose phototrophic biomass in the reaction zone, and while discharging evaluation purpose product from the reaction zone 10, wherein the evaluation purpose product includes a portion of the evaluation purpose phototrophic biomass, the process further includes:
(i) at least periodically measuring a process parameter to provide a plurality of measured values of the process parameter that have been measured during a time period (“at least periodically” means that the measuring could be done intermittently, at equally spaced intervals or at unequally spaced time intervals, or could be done continuously);
(ii) calculating molar growth rates of the evaluation purpose phototrophic biomass based on the plurality of measured values of the process parameter such that a plurality of molar growth rates of the evaluation purpose phototrophic biomass are determined during the time period; and
(iii) selecting a maximum molar growth rate from the determined plurality of molar growth rates of the evaluation purpose phototrophic biomass, such that the selected maximum molar growth rate is representative of the maximum molar growth rate of the production purpose phototrophic biomass within the reaction zone 10, and such that the maximum molar growth rate of the production purpose phototrophic biomass within the reaction zone is thereby provided.

In some embodiments, for example, the effected growth of the evaluation purpose phototrophic biomass in the reaction zone 10 is effected while the evaluation purpose reaction mixture is exposed to at least one evaluation purpose growth condition that is representative of a production purpose growth condition to which the production purpose reaction mixture is exposed to while growth of the production purpose phototrophic biomass in the reaction zone 10 is effected. In some embodiments, for example, the production purpose growth condition is any one of a plurality of production purpose growth conditions including composition of the reaction zone 10, reaction zone temperature, reaction zone pH, reaction zone light intensity, reaction zone lighting regimes, reaction zone lighting cycles, and reaction zone temperature. In some embodiments, for example, the discharging of the evaluation purpose product from the reaction zone 10 is effected as an overflow, and the overflow is effected in response to supplying of aqueous feed material to the reaction zone 10.

In another aspect, discharging of the product 58 is effected at a rate that matches the growth rate of the phototrophic biomass. In some embodiments, for example, this mitigates shocking of the phototrophic biomass in the reaction zone 10. With respect to some embodiments, for example, the discharging of the product 58 is controlled through the rate of supply of supplemental aqueous material supply 44, which influences the displacement from the photobioreactor 12 of the phototrophic biomass-comprising product 58 as an overflow 59 from the photobioreactor 12. In some of these embodiments, the upper portion of phototrophic biomass suspension in the reaction zone 10 overflows the photobioreactor 12 (for example, the phototrophic biomass is discharged through an overflow port of the photobioreactor 12) to provide the phototrophic biomass-comprising product 58. In other embodiments, for example, the discharging of the product 58 is controlled with a valve disposed in a fluid passage which is fluidly communicating with an outlet of the photobioreactor 12.

In some embodiments, for example, the discharging of the product 58 is effected continuously. In other embodiments, for example, the discharging of the product is effected periodically. In some embodiments, for example, the discharging of the product is designed such that the concentration of the biomass in the phototrophic biomass-comprising product 58 is maintained at a relatively low concentration. In those embodiments where the phototrophic biomass includes algae, it is desirable, for some embodiments, to effect discharging of the product 58 at lower concentrations to mitigate against sudden changes in the growth rate of the algae in the reaction zone 10. Such sudden changes could effect shocking of the algae, which thereby contributes to lower yield over the longer term. In some embodiments, where the phototrophic biomass is algae and, more specifically, Scenedesmus obliquus, the concentration of these algae in the phototrophic biomass-comprising product 58 could be between 0.5 and 3 grams per litre. The desired concentration of the discharged algae product 58 depends on the strain of algae such that this concentration range changes depending on the strain of algae. In this respect, in some embodiments, maintaining a predetermined water content in the reaction zone is desirable to promote the optimal growth of the phototrophic biomass, and this can also be influenced by controlling the supply of the supplemental aqueous material supply 44.

The phototrophic biomass-comprising product 58 includes water. In some embodiments, for example, the phototrophic biomass-comprising product 58 is supplied to a separator 52 for effecting removal of at least a fraction of the water from the phototrophic biomass-comprising product 58 to effect production of an intermediate concentrated phototrophic biomass-comprising product 34 and a recovered aqueous material 72 (generally, water). In some embodiments, for example, the separator 52 is a high speed centrifugal separator 52. Other suitable examples of a separator 52 include a decanter, a settling vessel or pond, a flocculation device, or a flotation device. In some embodiments, the recovered aqueous material 72 is supplied to a container 28, such as a container, for re-use by the process.

In some embodiments, for example, after the product 58 is discharged, and before being supplied to the separator 52, the phototrophic biomass-comprising product 58 is supplied to a harvest pond 54. The harvest pond 54 functions both as a buffer between the photobioreactor 12 and the separator 52, as well as a mixing vessel in cases where the harvest pond 54 receives different biomass strains from multiple photobioreactors. In the latter case, customization of a blend of biomass strains can be effected with a predetermined set of characteristics tailored to the fuel type or grade that will be produced from the blend.

As described above, the container 28 provides a source of supplemental aqueous material supply 44 for the reaction zone 10, and functions to contain the supplemental aqueous material supply 44 before supplemental aqueous material supply 44 is supplied to the reaction zone 10. Loss of water is experienced in some embodiments as moisture in the final phototrophic biomass-comprising product 36, as well as through evaporation in the dryer 32. The supplemental aqueous material in the container 28, which is recovered from the process, can be supplied to the reaction zone 10 as the supplemental aqueous material supply 44. In some embodiments, for example, the supplemental aqueous material supply 44 is supplied to the reaction zone 10 with the pump 281. In other embodiments, the supply can be effected by gravity, if the layout of the process equipment of the system, which embodies the process, permits. As described above, the supplemental aqueous material recovered from the process includes at least one of: (a) aqueous material 70 which has been condensed from the reaction zone feed material 22 while the reaction zone feed material 22 is being cooled before being supplied to the reaction zone 10, and (b) aqueous material 72 which has been separated from the phototrophic biomass-comprising product 58. In some embodiments, for example, the supplemental aqueous material supply 44 is supplied to the reaction zone 10 to influence overflow of the product 58 by increasing the upper level of the contents of the reaction zone 10. In some embodiments, for example, the supplemental aqueous material supply 44 is supplied to the reaction zone 10 to influence a desired predetermined concentration of phototrophic biomass to the reaction zone by diluting the contents of the reaction zone.

Examples of specific structures which can be used as the container 28 by allowing for containment of aqueous material recovered from the process, as above-described, include, without limitation, tanks, ponds, troughs, ditches, pools, pipes, tubes, canals, and channels.

In some embodiments, for example, the supplying of the supplemental aqueous material supply 44 to the reaction zone 10 is effected while the gaseous exhaust material 18 is being produced by the gaseous exhaust material producing process 20, and while the gaseous exhaust material reaction zone supply 24 is being supplied to the reaction zone feed material 22, and while the reaction zone feed material 22 is being supplied to the reaction zone 10. In some embodiments, for example, the exposing of the carbon dioxide-enriched phototrophic biomass disposed in the aqueous medium to photosynthetically active light radiation is effected while the supplying of the supplemental aqueous material supply to the reaction zone 10 is being effected.

In some embodiments, for example, when the upper level of the contents of the reaction zone 10 within the photobioreactor 12 becomes disposed below a predetermined minimum level, the initiation of the supply of, or an increase to the molar rate of supply of, the supplemental aqueous material supply 44 (which has been recovered from the process) is effected to the reaction zone 10. In some of these embodiments, for example, a level sensor 76 is provided for sensing the position of the upper level of the contents of the reaction zone 10 within the photobioreactor, and transmitting a signal representative of the upper level of the contents of the reaction zone 10 to the controller. Upon the controller comparing a received signal from the level sensor 76, which is representative of the upper level of the contents of the reaction zone 10, to a predetermined low level value, and determining that the sensed upper level of the contents of the reaction zone is below the predetermined low level value, the controller effects the initiation of the supply of, or an increase to the molar rate of supply of, the supplemental aqueous material supply 44. When the supply of the supplemental aqueous material supply 44 to the reaction zone 10 is effected by a pump 281, the controller actuates the pump 281 to effect the initiation of the supply of, or an increase to the rate of supply of, the supplemental aqueous material supply 44 to the reaction zone 10. When the supply of the supplemental aqueous material supply 44 to the reaction zone 10 is effected by gravity, the controller actuates the opening of a control valve to effect the initiation of the supply, or an increase to the molar rate of supply of, the supplemental aqueous material supply 44 to the reaction zone 10. For example, control of the position of the upper level of the contents of the reaction zone 10 is relevant to operation for some of those embodiments where harvesting is effected from a lower portion of the reaction zone 10. In those embodiments where harvesting is effected by an overflow, in some of these embodiments, control of the position of the upper level of the contents of the reaction zone 10 is relevant during the “seeding stage” of operation of the photobioreactor 12.

In some embodiments, supply of the supplemental aqueous material supply 44 to the reaction zone 10 is dictated by algae concentration. In this respect, molar algae concentration in the reaction zone is sensed by a cell counter, such as the cell counters described above. The sensed molar algae concentration is transmitted to the controller, and when the controller determines that the sensed molar algae concentration exceeds a predetermined high algae concentration value, the controller responds by actuating the pump 281 to effect supply of the supplemental aqueous material supply 44 to the reaction zone 10.

In some embodiments, for example, where the discharging of the product 58 is controlled with a valve disposed in a fluid passage which is fluidly communicating with an outlet of the photobioreactor 12, molar concentration of algae in the reaction zone is sensed by a cell counter 47, such as the cell counters described above. The sensed molar concentration of algae is transmitted to the controller, and when the controller determines that the sensed molar algae concentration exceeds a predetermined high molar algae concentration value, the controller responds by actuating opening of the valve to effect an increase in the molar rate of discharging of the product 58 from the reaction zone 10.

In some embodiments, for example, a source of additional make-up water 68 is provided to mitigate against circumstances when the supplemental aqueous material supply 44 is insufficient to make-up for water which is lost during operation of the process. In this respect, in some embodiments, for example, the supplemental aqueous material supply 44 is mixed with the reaction zone feed material 22 in the sparger 40. Conversely, in some embodiments, for example, accommodation for draining of the container 28 to drain 66 is provided to mitigate against the circumstances when aqueous material recovered from the process exceeds the make-up requirements.

In some embodiments, for example, a reaction zone gaseous effluent product 80 is discharged from the reaction zone 10. At least a fraction of the reaction zone gaseous effluent 80 is recovered and supplied to a reaction zone 110 of a combustion process unit operation 100. As a result of the photosynthesis being effected in the reaction zone 10, the reaction zone gaseous effluent 80 is rich in oxygen relative to the gaseous exhaust material reaction zone supply 24. The gaseous effluent 80 is supplied to the combustion zone 110 of a combustion process unit operation 100 (such as a combustion zone 110 disposed in a reaction vessel), and, therefore, functions as a useful reagent for the combustion process being effected in the combustion process unit operation 100. The reaction zone gaseous effluent 80 is contacted with combustible material (such as carbon-comprising material) in the combustion zone 100, and a reactive process is effected whereby the combustible material is combusted. Examples of suitable combustion process unit operations 100 include those in a fossil fuel-fired power plant, an industrial incineration facility, an industrial furnace, an industrial heater, an internal combustion engine, and a cement kiln.

In some embodiments, for example, the contacting of the recovered reaction zone gaseous effluent 80 with a combustible material is effected while the gaseous exhaust material 18 is being produced by the gaseous exhaust material producing process 20. In some embodiments, for example, the contacting of the recovered reaction zone gaseous effluent with a combustible material is effected while the gaseous exhaust material reaction zone supply 24 is being supplied to the reaction zone feed material 22. In some embodiments, for example, the contacting of the recovered reaction zone gaseous effluent with a combustible material is effected while the reaction zone feed material is being supplied to the reaction zone. In some embodiments, for example, the exposing of the carbon dioxide-enriched phototrophic biomass disposed in the aqueous medium to photosynthetically active light radiation is effected while the contacting of the recovered reaction zone gaseous effluent with a combustible material is being effected.

The intermediate concentrated phototrophic biomass-comprising product 34 is supplied to a dryer 32 which supplies heat to the intermediate concentrated phototrophic biomass-comprising product 34 to effect evaporation of at least a fraction of the water of the intermediate concentrated phototrophic biomass-comprising product 34, and thereby effect production of a final phototrophic biomass-comprising product 36. As discussed above, in some embodiments, the heat supplied to the intermediate concentrated phototrophic biomass-comprising product 34 is provided by a heat transfer medium 30 which has been used to effect the cooling of the reaction zone feed material 22 prior to supply of the reaction zone feed material 22 to the reaction zone 10. By effecting such cooling, heat is transferred from the reaction zone feed material 22 to the heat transfer medium 30, thereby raising the temperature of the heat transfer medium 30. In such embodiments, the intermediate concentrated phototrophic biomass-comprising product 34 is at a relatively warm temperature, and the heat requirement to effect evaporation of water from the intermediate concentrated phototrophic biomass-comprising product 34 is not significant, thereby rendering it feasible to use the heated heat transfer medium 30 as a source of heat to effect the drying of the intermediate concentrated phototrophic biomass-comprising product 34. As discussed above, after heating the intermediate concentrated phototrophic biomass-comprising product 34, the heat transfer medium 30, having lost some energy and becoming disposed at a lower temperature, is recirculated to the heat exchanger 26 to effect cooling of the reaction zone feed material 22. The heating requirements of the dryer 32 are based upon the rate of supply of intermediate concentrated phototrophic biomass-comprising product 34 to the dryer 32. Cooling requirements (of the heat exchanger 26) and heating requirements (of the dryer 32) are adjusted by the controller to balance the two operations by monitoring flow rates and temperatures of each of the reaction zone feed material 22 and the rate of production of the product 58 through discharging of the product 58 from the photobioreactor.

In some embodiments, changes to the phototrophic biomass growth rate effected by changes to the rate of supply of the gaseous exhaust material reaction zone supply 24 to the reaction zone material feed 22 are realized after a significant time lag (for example, in some cases, more than three (3) hours, and sometimes even longer) from the time when the change is effected to the rate of supply of the gaseous exhaust material reaction zone supply 24 to the reaction zone feed material 22. In comparison, changes to the thermal value of the heat transfer medium 30, which are based on the changes in the rate of supply of the gaseous exhaust material reaction zone supply 24 to the reaction zone feed material 22, are realized more quickly. In this respect, in some embodiments, a thermal buffer is provided for storing any excess heat (in the form of the heat transfer medium 30) and introducing a time lag to the response of the heat transfer characteristics of the dryer 32 to the changes in the gaseous exhaust material reaction zone supply 24. In some embodiments, for example, the thermal buffer is a heat transfer medium storage tank. Alternatively, an external source of heat may be required to supplement heating requirements of the dryer 32 during transient periods of supply of the gaseous exhaust material reaction zone supply 24 to the reaction zone material 22. The use of a thermal buffer or additional heat may be required to accommodate changes to the rate of growth of the phototrophic biomass, or to accommodate start-up or shutdown of the process. For example, if growth of the phototrophic biomass is decreased or stopped, the dryer 32 can continue operating by using the stored heat in the buffer until it is consumed, or, in some embodiments, use a secondary source of heat.

Further embodiments will now be described in further detail with reference to the following non-limitative example.

### EXAMPLE 1

A prophetic example, exemplifying an embodiment of determining a target value of a process parameter (algae concentration in the reaction zone of a photobioreactor), and effecting operation of an embodiment of the above-described process, including modulating the molar rate of discharge of the phototrophic biomass-comprising product from the reaction zone based on a deviation of a sensed value of the process parameter from the target value, will now be described.

Initially, an initial algae concentration in an aqueous medium, with suitable nutrients, is provided in a reaction zone of a photobioreactor. Gaseous carbon dioxide is supplied to the reaction zone, and the reaction zone is exposed to light from a light source (such as LEDs), to effect growth of the algae. When algae concentration in the reaction zone reaches 0.5 grams per litre, water is flowed to the reaction zone of the photobioreactor to effect harvesting of the algae by effecting overflow of the reactor contents, and an initial target algae concentration is set at 0.5 grams per litre. Initially, the supplied water is flowed at a relatively moderate and constant rate such that the half (½) of the volume of the photobioreactor is exchanged per day, as it is found that periodically replacing water volume within the reaction zone with fresh water promotes growth of the algae and enables attaining the target value in a shorter period of time. If the algae growth rate is lower than the dilution rate, and the sensed algae concentration drops at least 2% from the algae concentration set point at any time during this determination exercise, the control system will stop or reduce the dilution rate to avoid further dilution of the algae concentration in the reaction zone. If the algae growth rate is higher than the dilution rate, the algae concentration will increase above the initial algae concentration set point, and the control system will increase the algae concentration set point so as to keep pace with the increasing algae concentration, while maintaining the same dilution rate. For example, the algae concentration may increase to 0.52 grams per litre, at which point the control system will increase the algae concentration set point to 0.51. The control system continues to monitor the increase in algae concentration and, in parallel, increasing the target algae concentration. When a maximum change in the algae growth rate has been detected, the target algae concentration is locked at its existing value to become the target value, and dilution rate is then modulated so that harvesting of the algae is effected at a rate which is equivalent to the growth rate of the algae within the photobioreactor when the algae concentration is at the target value.

Algae growth rate corresponds with algae concentration. When a considerable change in the algae growth rate is detected, this is indicative of growth of algae within the reaction zone at, or close to, its maximum rate, and this growth rate corresponds to an algae concentration at the target value. In this respect, by maintaining algae concentration in the reaction zone at the target value by controlling dilution rate, algae growth is maintained at or close to the maximum, and, as a corollary, over time, the rate of discharge of algae is optimized.

In the above description, for purposes of explanation, numerous details are set forth in order to provide a thorough understanding of the present disclosure. However, it will be apparent to one skilled in the art that these specific details are not required in order to practice the present disclosure. Although certain dimensions and materials are described for implementing the disclosed example embodiments, other suitable dimensions and/or materials may be used within the scope of this disclosure. All such modifications and variations, including all suitable current and future changes in technology, are believed to be within the sphere and scope of the present disclosure. All references mentioned are hereby incorporated by reference in their entirety.

What is claimed is:

1. A process for growing a phototrophic biomass in a reaction zone, wherein the reaction zone comprises a production purpose reaction mixture that is operative for effecting photosynthesis upon exposure to photosynthetically active light radiation, wherein the production purpose reaction mixture comprises production purpose phototrophic biomass that is operative for growth within the reaction zone, wherein the growth of the production purpose phototrophic biomass comprises that which is effected by the photosynthesis, comprising:
while effecting growth of the production purpose phototrophic biomass in the reaction zone, and while discharging reaction zone product from the reaction zone, wherein the reaction zone product comprises a portion of the production purpose phototrophic biomass:
when a sensed value of a process parameter is different than a target value of the process parameter, modulating the molar rate of discharge of the reaction zone product from the reaction zone, wherein the target value of the process parameter is based upon a desired molar growth rate of the production purpose phototrophic biomass within the reaction zone.

2. The process as claimed in claim 1, wherein the target value of the process parameter corresponds to a desired molar growth rate of the production purpose phototrophic biomass.

3. The process as claimed in claim 1, wherein the modulating is effected in response to comparing of the sensed value of the process parameter to the target value of the process parameter.

4. The process as claimed in claim 3, further comprising sensing a process parameter to provide the sensed value of the process parameter.

5. The process as claimed in claim 1, wherein the sensed value of the process parameter is representative of the reaction zone concentration of the production purpose phototrophic biomass.

6. The process as claimed in claim 5, wherein the sensed value of the process parameter is the reaction zone concentration of the production purpose phototrophic biomass.

7. The process as claimed in claim 5, wherein the sensed value of the process parameter is the concentration of the production purpose phototrophic biomass in the reaction zone product.

8. The process as claimed in claim 1, wherein the target value is predetermined.

9. The process as claimed in claim 8, wherein the desired growth rate is predetermined.

10. The process as claimed in claim 1, wherein the desired growth rate is predetermined.

11. The process as claimed in claim 1, wherein the effected growth of the production purpose phototrophic biomass in the reaction zone is being effected within 10% of the desired growth rate.

12. The process as claimed in claim 1, wherein the effected growth of the production purpose phototrophic biomass in the reaction zone is being effected within 5% of the desired growth rate.

13. The process as claimed in claim 1, wherein the effected growth of the production purpose phototrophic biomass in the reaction zone is being effected within 1% of the desired growth rate.

14. The process as claimed in claim 8, wherein the predetermination of the target value comprises:
providing an evaluation purpose reaction mixture that is representative of the production purpose reaction mixture and is operative for effecting photosynthesis upon exposure to photosynthetically active light radiation, such that the phototrophic biomass of the evaluation purpose reaction mixture is an evaluation purpose phototrophic biomass that is representative of the production purpose phototrophic biomass; and
while effecting growth of the evaluation purpose phototrophic biomass in the reaction zone, and while discharging evaluation purpose product from the reaction zone, wherein the evaluation purpose product comprises a portion of the evaluation purpose phototrophic biomass;
at least periodically measuring the process parameter to provide a plurality of measured values of the process parameter that have been measured during a time period;
calculating molar growth rates of the evaluation purpose phototrophic biomass based on the plurality of measured values of the process parameter such that a plurality of molar growth rates of the evaluation purpose phototrophic biomass are determined during the time period; and
establishing a relationship between the molar growth rate of the evaluation purpose phototrophic biomass and the process parameter, based on the calculated molar growth rates and the measured values of the process parameter upon which the calculated molar growth rates have been based;
such that the established relationship between the molar growth rate of the evaluation purpose phototrophic biomass and the process parameter is representative of a relationship between the molar growth rate of the production purpose phototrophic biomass within the reaction zone and the process parameter, and such that the relationship between the molar growth rate of the production purpose phototrophic biomass within the reaction zone and the process parameter is thereby provided; and
based on the relationship between the molar growth rate of the production purpose phototrophic biomass within the reaction zone and the process parameter, selecting the desired molar growth rate, and determining the target value as being the process parameter at which the desired molar growth rate is being effected, such that the correspondence between the target value and the desired molar growth rate is also thereby effected.

15. The process as claimed in claim 14, wherein the effected growth of the evaluation purpose phototrophic biomass in the reaction zone is effected while the evaluation purpose reaction mixture is exposed to at least one evaluation purpose growth condition that is representative of a production purpose growth condition to which the production purpose reaction mixture is exposed to while growth of the production purpose phototrophic biomass in the reaction zone is being effected.

16. The process as claimed in claim 15, wherein the production purpose growth condition is any one of a plurality of production purpose growth conditions including composition of the reaction zone, reaction zone temperature, reaction zone pH, reaction zone light intensity, reaction zone lighting regimes, reaction zone lighting cycles, and reaction zone temperature.

17. The process as claimed in claim 14, wherein the desired molar growth rate is at least 90% of the maximum molar growth rate of the production purpose phototrophic biomass.

18. The process as claimed in claim 15, wherein the desired molar growth rate is at least 99% of the maximum growth rate of the phototrophic biomass.

19. The process as claimed in claim 1, wherein the reaction zone is disposed within a photobioreactor, and wherein the reaction zone product comprises an overflow that is discharged from the photobioreactor, and the overflow is effected by supplying of an aqueous feed material to the reaction zone.

20. The process as claimed in claim 1, wherein the modulating of the molar rate of discharge of the reaction zone product from the reaction zone is effected by modulating the molar rate of supply of the aqueous feed material to the reaction zone.

21. The process as claimed in claim 1, wherein, when a sensed value of a process parameter is less than a target value of the process parameter, the modulating comprises effecting a decrease in the molar rate of discharge of the reaction zone product from the reaction zone.

22. The process as claimed in claim 21, wherein the modulating is effected by effecting a decrease in the molar rate of supply of the aqueous feed material to the reaction zone.

23. The process as claimed in claim 22, wherein the reaction zone product that is discharged from the reaction zone comprises an overflow from a photobioreactor, and the overflow is effected by supplying of an aqueous feed material to the reaction zone.

24. The process as claimed in claim 1, wherein, when a measured value of a process parameter is greater than a target value of the process parameter, the modulating comprises effecting an increase in the molar rate of discharge of the reaction zone product from the reaction zone.

25. The process as claimed in claim 24, wherein the modulating is effected by effecting an increase in the rate of supply of the aqueous feed material to the reaction zone.

26. The process as claimed in claim 25, wherein the reaction zone product that is discharged from the reaction zone comprises an overflow from a photobioreactor, and the overflow is effected by supplying of an aqueous feed material to the reaction zone.

27. The process as claimed in claim 19, wherein the aqueous feed material comprises substantially no phototrophic biomass.

28. The process as claimed in claim 19, wherein the aqueous feed material comprises phototrophic biomass at a concentration less than the reaction zone concentration of phototrophic biomass.

29. The process as claimed in claim 1, wherein the effecting of the growth of the phototrophic biomass comprises supplying carbon dioxide to the reaction zone and exposing the production purpose reaction mixture to photosynthetically active light radiation.

30. The process as claimed in claim 29, wherein the carbon dioxide is supplied while the growth is being effected.

31. The process as claimed in claim 30, wherein at least a fraction of the carbon dioxide being supplied to the reaction zone is supplied from a gaseous exhaust material while the gaseous exhaust material is being discharged from a gaseous exhaust material producing process.

32. The process as claimed in claim 1, wherein the production purpose reaction mixture further comprises water and carbon dioxide.

33. The process as claimed in claim 14, wherein the production purpose reaction mixture further comprises water and carbon dioxide; and the evaluation purpose reaction mixture further comprises water and carbon dioxide.

34. A process for growing a phototrophic biomass in a reaction zone, wherein the reaction zone comprises a production purpose reaction mixture that is operative for effecting photosynthesis upon exposure to photosynthetically active light radiation, wherein the production purpose reaction mixture comprises production purpose phototrophic biomass that is operative for growth within the reaction zone, wherein the growth of the production purpose phototrophic biomass comprises growth which is effected by the photosynthesis, comprising:
while effecting growth of the production purpose phototrophic biomass within the reaction zone at a desired molar growth rate, discharging a reaction zone product including production purpose phototrophic biomass from the reaction zone to provide a molar rate of discharge of the production purpose phototrophic biomass that is within 10% of the desired molar growth rate;
wherein the desired molar growth rate is at least 90% of the maximum growth rate of the production purpose phototrophic biomass within the reaction zone.

35. The process as claimed in claim 34, wherein the reaction zone is disposed within a photobioreactor, and wherein the reaction zone product is discharged from the reaction zone and comprises an overflow from the photobioreactor.

36. The process as claimed in claim 34, wherein the effecting of the growth of the production purpose phototrophic biomass comprises supplying carbon dioxide to the reaction zone and exposing the production purpose reaction mixture to photosynthetically active light radiation.

37. The process as claimed in claim 34, wherein the desired molar growth rate is at least 95% of the maximum growth rate of the production purpose phototrophic biomass within the reaction zone.

38. The process as claimed in claim 34, wherein the desired molar growth rate is at least 99% of the maximum growth rate of the production purpose phototrophic biomass within the reaction zone.

39. The process as claimed in claim 34, wherein the molar rate of discharge of the production purpose phototrophic biomass that is provided is within 5% of the desired molar growth rate.

40. The process as claimed in claim 34, wherein the molar rate of discharge of the production purpose phototrophic biomass that is provided is within 1% of the desired molar growth rate.

41. The process as claimed in claim 34, wherein the maximum growth rate of the production purpose phototrophic biomass is predetermined;
and wherein the predetermination of the maximum molar growth rate of the production purpose phototrophic biomass comprises providing an evaluation purpose reaction mixture that is representative of the production purpose reaction mixture and is operative for effecting photosynthesis upon exposure to photosynthetically active light radiation, such that the phototrophic biomass of the evaluation purpose reaction mixture is an evaluation purpose phototrophic biomass that is representative of the production purpose phototrophic biomass; and
while effecting growth of the evaluation purpose phototrophic biomass in the reaction zone, and while discharging evaluation purpose product from the reaction zone, wherein the evaluation purpose product comprises a portion of the evaluation purpose phototrophic biomass:
at least periodically measuring a process parameter to provide a plurality of measured values of the process parameter that have been measured during a time period;
calculating molar growth rates of the evaluation purpose phototrophic biomass based on the plurality of measured values of the process parameter such that a plurality of molar growth rates of the evaluation purpose phototrophic biomass are determined during the time period; and
selecting a maximum molar growth rate from the determined plurality of molar growth rates of the evaluation purpose phototrophic biomass, such that the selected maximum molar growth rate is representative of the maximum molar growth rate of the production purpose phototrophic biomass within the reaction zone, and such that the maximum molar growth rate of the production purpose phototrophic biomass within the reaction zone is thereby provided.

42. The process as claimed in claim 31, wherein the effected growth of the evaluation purpose phototrophic biomass in the reaction zone is effected while the evaluation purpose reaction mixture is exposed to at least one evaluation purpose growth condition that is representative of a production purpose growth condition to which the production purpose reaction mixture is exposed to while growth of the production purpose phototrophic biomass in the reaction zone is effected.

43. The process as claimed in claim 42, wherein the production purpose growth condition is any one of a plurality of production purpose growth conditions including composition of the reaction zone, reaction zone temperature, reaction zone pH, reaction zone light intensity, reaction zone lighting regimes, reaction zone lighting cycles, and reaction zone temperature.

44. The process as claimed in claim 34, wherein the reaction zone product is discharged while aqueous feed material is being supplied to the reaction zone and reaction zone product is being discharged from the reaction zone.

45. The process as claimed in claim 44, wherein the aqueous feed material comprises substantially no production purpose phototrophic biomass.

46. The process as claimed in claim 42, wherein the aqueous feed material comprises production purpose phototrophic biomass at a concentration less than the reaction zone concentration of the production purpose phototrophic biomass.

47. The process as claimed in claim 36, wherein the carbon dioxide is supplied while the growth is being effected, and wherein at least a fraction of the carbon dioxide being supplied to the reaction zone is supplied from a gaseous exhaust material while the gaseous exhaust material is being discharged from a gaseous exhaust material producing process.

48. A process for growing a phototrophic biomass in a reaction zone, wherein the reaction zone comprises a production purpose reaction mixture that is operative for effecting photosynthesis upon exposure to photosynthetically active light radiation, wherein the production purpose reaction mixture comprises production purpose phototrophic biomass that is operative for growth within the reaction zone, wherein the growth of the production purpose phototrophic biomass comprises growth which is effected by the photosynthesis, comprising:
while effecting growth of the production purpose phototrophic biomass within the reaction zone at a desired molar growth rate, discharging a reaction zone product including production purpose phototrophic biomass from the reaction zone to provide a molar rate of discharge of the production purpose phototrophic biomass that is equivalent to a desired molar growth rate of the production purpose phototrophic biomass within the reaction zone.

49. The process as claimed in claim 48, wherein the desired molar growth rate is equivalent to the maximum growth rate of the production purpose phototrophic biomass within the reaction zone.
